(19) United States
(12) Patent Application Publication
(10) Pub. No.: US 2012/0064135 A1
(43) Pub. Date: Mar. 15, 2012

(54) Benzoyl Peroxide Composition, Methods for Making Same, and Pharmaceutical or Cosmetic Formulations Comprising Same, and Uses Thereof

(75) Inventors: Daniel Levin, La Canada, CA (US); Arthur Harms, Pasadena, CA (US)

(73) Assignee: Norac Pharma, Azusa, CA (US)

(21) Appl. No.: 13/210,855

(22) Filed: Aug. 16, 2011

Related U.S. Application Data
(60) Provisional application No. 61/389,456, filed on Oct. 4, 2010.
(60) Provisional application No. 61/383,149, filed on Sep. 15, 2010.

(51) Int. Cl. A61K 8/11; A61K 8/38; A61K 9/00; A61K 31/4436; A61K 31/203; A61K 31/7056; A61K 31/7048; A61K 31/136; A61K 31/192; A61K 47/44; A61P 31/00; A61P 31/10; A61P 31/12; A61P 35/00; A61Q 19/00; A61P 31/04; A61P 17/00; A61P 17/10; A61K 31/327

(52) U.S. Cl. 424/401; 514/714; 424/400; 514/337; 514/559; 514/24; 514/29; 514/646; 514/569; 514/772

(57) ABSTRACT

The present invention relates to the preparation of compositions comprising benzoyl peroxide, with or without other additional active ingredients. The process involves introducing benzoyl peroxide, along with any other active ingredients present, into a fatty substance that contains and protects the ingredients that would otherwise be unstable when in contact with one another. The composition is designed to allow all ingredients to become available for skin contact or skin absorption when the fatty substance softens and/or melts as the composition is applied to the skin. The benzoyl peroxide may be pre-micronized to a particle distribution size of about d₉₀ of 0.1 to 150 microns, preferably d₉₀ of 10 to 15 microns. Further, pharmaceutical or cosmetic ingredients may be contained within the fatty substances, with or without, benzoyl peroxide therein or may be present outside of the fatty substance but elsewhere within formulated pharmaceutical or cosmetic products using the active ingredients protected by the fatty substance. These compositions are useful in aqueous-based formulations to treat diseases by topical, transdermal and/or subcutaneous administration.

### CROSS REFERENCE TO RELATED APPLICATIONS

This application claims the benefit of priority of U.S. Provisional Application No. 61/389,456, filed Oct. 15, 2010, and U.S. Provisional Application No. 61/383,149, filed Sep. 27, 2010, each of which is incorporated herein by reference.

### BACKGROUND

Benzoyl peroxide is an ingredient of many products used for the treatment of skin disorders such as acne, seborrhea, and skin infections and is often coformulated with other pharmaceutical active ingredients in skin care products, including antibiotics (such as clindamycin or erythromycin) and retinoids such as adapalene. However, other active pharmaceutical ingredients such as tazarotene, tretinoin, and sulfur that would be useful in coformulation with benzoyl peroxide are currently not formulated with benzoyl peroxide due to chemical incompatibilities between benzoyl peroxide and these other active pharmaceutical ingredients. As such, it is a goal of the present invention to provide compositions of benzoyl peroxide suitable for coformulation with other pharmaceutical active ingredients that would otherwise be incompatible with benzoyl peroxide, coapplied with not only pharmaceutical ingredients but also with cosmetic ingredients such as hyaluronic acid (a cosmetic hydrating agent) and other materials. Such coformulations provide beneficial properties of the combined active ingredients without the negative interactions of benzoyl peroxide and the other active ingredients.

Many active ingredients in skin care products cannot be easily formulated with, or are incompatible with, benzoyl peroxide. In fact, many formulations of benzoyl peroxide and other active ingredients have a relatively short shelf life and often need to be stored in a cold environment to mitigate decomposition of the co-active ingredient and/or the benzoyl peroxide. For example, BENZACLIN topical gel, a coformulation of benzoyl peroxide with clindamycin antibiotic for the treatment of acne, has a shelf life of only 3 months at 25° C. BENZAMYCIN, a coformulation of benzoyl peroxide with erythromycin antibiotic for the treatment of acne, has a shelf life of only 3 months under refrigerated storage conditions of 2-8° C. The instability of such formulations has been attributed to reactions between benzoyl peroxide and the co-ingredient(s), resulting in progressive degradation of the active components and/or the formulation as a whole, as well as the generation of unwanted by-products. As such, a mechanism and formulation to prevent and/or mitigate the adverse reactions between benzoyl peroxide and other active ingredients in pharmaceutical and/or cosmetic formulations is sorely needed. The present invention provides a solution to this problem. Incorporation of benzoyl peroxide and/or other co-ingredients within a fatty substance mitigates the reactions between the coformulated active ingredients and thereby improves the stability and increase shelf life of the resultant formulation.

There is therefore, a need to provide stable formulations containing benzoyl peroxide and retinoids or other active ingredients in such a way as to protect the active ingredients from one another during drug product manufacture and storage, whilst nevertheless allowing release of or exposure to the active ingredients on skin application. In fact, there is a need to formulate benzoyl peroxide or other active ingredients with other coactive ingredients that may not be compatible with the benzoyl peroxide or other active ingredients to treat many skin infections, including bacterial infections, yeast skin infections, fungal skin infections or skin disorders caused by virus infection such as warts or even cancer of the skin or underlying tissue.

The following provide examples of attempts to formulate benzoyl peroxide.

Attempts have been made to provide stable formulations of benzoyl peroxide and other active co-ingredients. For instance, WO 01/80823 describes the preparation of sol-gel microcapsules suitable for encapsulating benzoyl peroxide and other active ingredients such as erythromycin or other anti-acne medication in a hard silica capsule. However the sol-gel microcapsules prepared by this procedure require either dehydration or a significant force to fully release the active ingredients onto the skin making them inefficient for topical medication release. In addition, the chemicals required to prepare the sol-gel microcapsules have potential safety concerns.

U.S. Pat. No. 7,754,240 and US 2008/0166413 formulate benzoyl peroxide with cocoa butter, but do not achieve the formulations of the present invention. First, the formulations disclosed therein all require unit dosages of the active ingredients. Further, the formulations require that they all be in tablet form for application, whereas the present invention does not. The formulations described in these documents are not designed to protect benzoyl peroxide and other coformulated active ingredients from interacting with another and degrading one another and indeed the processes for preparing the pharmaceutical compositions that are described in U.S. Pat. No. 7,754,240 and US 2008/0166413 comprise refrigeration to harden, followed by breaking down and granulating the frozen solid so that if benzoyl peroxide were present it would be exposed on granulation which would result in mutual degradation in the event of coformulation with other incompatible active ingredients.

U.S. Pat. No. 5,409,764 describes encapsulation of benzoyl peroxide by polyurethane resin capsules prepared from toluene diisocyanate (TDI) and tetraethylenepentamine. However, TDI is classified as an “extremely hazardous substance” and has toxicological properties that make it unsuitable for pharmaceutical and cosmetic preparations. Furthermore, this patent does not describe the means to segregate and protect benzoyl peroxide and other mutually incompatible pharmaceutical active ingredients from one another.

US Patent Application 2007/0190124 describes the preparation of an adhesive film containing benzoyl peroxide prepared by solvent evaporation, which is developed as a drug delivery system to effect slow release of benzoyl peroxide and other active ingredients by diffusion at and through the skin. However, release of the active ingredients is limited by the rate of diffusion from the adhesive film. In addition, the process requires evaporation of volatile solvents to generate the film and is thus incompatible with many skincare formulations. Furthermore, this patent does not describe the means to segregate and protect benzoyl peroxide and other mutually incompatible pharmaceutical active ingredients from one another.

WO 2010/041141 describes the preparation of foam formulations including formulations containing benzoyl peroxide with triglycerides. However, such formulations do not prevent benzoyl peroxide from interacting with other coformulated active ingredients. Similarly, US 2003/0170196 describes the formulation of benzoyl peroxide or retinoid active ingredients with polyacrylamide gelling agents. Again, however, this patent does not describe the means to segregate and protect benzoyl peroxide and other mutually incompatible pharmaceutical and cosmetically active ingredients from one another and the interaction of benzoyl peroxide and retinoids would not be adequately mitigated by the gel formation.

U.S. Pat. Nos. 3,957,971 and 5,874,105 describe liposomes as delivery systems. However, such systems do not allow for the easy segregation of benzoyl peroxide from other active ingredients nor do they allow for easy release of the active ingredients onto the skin. U.S. Pat. No. 5,851,538 describes a commercially available protection system based on the adsorption of active ingredient(s) in pores that are present in a sponge form of an organic polymer. However, such a protection system would not provide an appropriate diffusion barrier to prevent active ingredients from diffusing out of the sponge and/or to prevent other materials from diffusing into the sponge on storage of drug products prepared using such a system. As such, this patent does not describe the means to segregate and protect benzoyl peroxide and other mutually incompatible pharmaceutical active ingredients from one another, nor would active ingredients necessarily be rapidly or efficiently released upon skin application.

U.S. Pat. No. 6,171,600 describes emulsions and double-emulsions containing an active substance for use in various medical applications. However, the processes described in U.S. Pat. No. 6,171,600 do not provide for the preparation or isolation of encapsulated or solubilized benzoyl peroxide compositions suitable for subsequent formulation into benzoyl peroxide containing pharmaceutical or cosmetic compositions to achieve separation of the benzoyl peroxide from other active components that would otherwise react with the benzoyl peroxide with mutual degradation.

There is therefore the need to provide compositions to allow segregation and protection of benzoyl peroxide from other pharmaceutical and/or cosmetically active ingredients to avoid them interacting adversely with one another during product manufacture or storage.

This need also applies to segregation and protection of other pharmaceutical and/or cosmetically active ingredients to avoid them interacting adversely with one another during product manufacture or storage. The present invention unexpectedly has been found to fulfill this need to segregate pharmaceutical and/or cosmetic ingredients.

Furthermore, it also is beneficial to control the particle size distribution of both the fatty substances, such as cocoa butter, of the present invention, used in formulations to deliver pharmaceutical and/or cosmetic ingredients, and the incorporated pharmaceutical and/or cosmetic active ingredient(s) within the cocoa butter or other fatty substance where those incorporated pharmaceutical active ingredient(s) and/or cosmetic ingredients are not homogeneously dissolved into the cocoa butter or other fatty substances and therefore need to be better delivered by controlling their particle sizes. This need is addressed by the present invention.

In pharmaceutical applications, the size of the benzoyl peroxide particles within the cocoa butter or other fatty substance(s) can have an impact on the intended efficacy of the benzoyl peroxide in the final application. This need is addressed by the present invention.

In therapeutic applications, such as in the treatment of acne, the particle size of both benzoyl peroxide in various formulations such as creams, gels, lotions and other formulations, can greatly affect the efficacy of the product. Indeed, there appears to be a particle size distribution within which the particle size of benzoyl peroxide is ideally suited for acne treatment and other skin conditions. The particle size distribution is characterized by a d₉₀ from 0.1 to 150 microns, more preferably by a d₉₀ from 5 to 25 microns, and most preferably by a d₉₀ from 10 to 15 microns. Particle sizes within these specified ranges allow the benzoyl peroxide to be more thoroughly and more beneficially dispersed into the product, thereby making it easier to distribute the benzoyl peroxide particles evenly across the area of application and to be more easily introduced into the affected pores of the skin for treatment. Additionally, the use of benzoyl peroxide particles within these preferred ranges leads to a less gritty product in terms of skin feel by the end-user, resulting in higher patient usage compliance. This control of benzoyl peroxide particle size is of value to ensure efficient coating of the benzoyl peroxide when the benzoyl peroxide is dispersed within a fatty substance to prevent adverse reaction of the benzoyl peroxide with other pharmaceutical or cosmetic ingredients present in the drug product or cosmetic product using the benzoyl peroxide and/or other active ingredient or ingredients present in the fatty substance. This need is addressed by the present invention.

The particle size distribution of the particles of cocoa butter or other fatty substance in which the benzoyl peroxide or other pharmaceutical active ingredient(s) is dispersed are also important for pharmaceutical preparations both for aesthetic reasons and for efficacy, in that very large particles of the cocoa butter or other fatty substance can be hard to formulate consistently with other ingredients for a pharmaceutical drug product. Also, without control of particle size distribution, the resulting lumpy consistency and uneven distribution of the pharmaceutically active ingredient(s) can result in inefficient treatment of acne or other skin disorders due to variable concentration of pharmaceutically active ingredient(s) over the surface of the skin to which such products are applied. Similarly, very small particles of the cocoa butter or other fatty substance can compromise the efficiency with which the pharmaceutically active ingredients are incorporated within the cocoa butter or other fatty substance such that those ingredients are not efficiently segregated from other components in the drug product formulations in which they are used. The particle size distribution of benzoyl peroxide in a fatty substance having a melting temperature of about 25° C. to about 45° C. in various formulations such as creams, gels, lotions and other formulations, can affect the efficacy of the product. The particle size distribution of the fatty substance having a melting temperature of about 25° C. to about 45° C. in which the benzoyl peroxide or other Active Pharmaceutical Ingredient (API) is dispersed, is ideally suited for treatment of acne and other skin diseases when the particle size distribution of the fatty substance particles is characterized by a d₉₀ from 0.1 to 200 microns, more preferably by a d₉₀ from 10 to 50 microns, and most preferably by a d₉₀ from 15 to 25 microns. Particle sizes of the fatty substance into which the API is dispersed within these specified ranges allow the active ingredient to be more thoroughly and more beneficially dispersed into the formulated product to achieve a more even distribution of the benzoyl peroxide or other API across the area of application and also to more easily introduce the active ingredient into affected pores of the skin for treatment of acne or other conditions. This need is addressed by the present invention.

As such, the compositions of the present invention may have the benzoyl peroxide and fatty substance particles optionally further reduced or micronized in the interest of increasing bioavailability.

Also, it is important to ensure that the particle size distribution of the benzoyl peroxide within the fatty substance is controlled in such a way as to ensure that the benzoyl peroxide particles are substantially contained within the fatty substance and that the benzoyl peroxide is substantially dispersed and retained within the fatty substance in order to minimize adverse interactions with other coformulated active ingredients outside of the fatty substance in the product formulation containing the benzoyl peroxide, or to minimize adverse interactions with other coformulated active ingredients in other particles of the fatty substance, or to minimize adverse interactions with other coformulated active ingredients in the same particles of the fatty substance, or to minimize adverse interactions between other active ingredients besides benzoyl peroxide that equally may be dispersed within particles of the fatty substance. This need is addressed by the present invention.

The methods used to manufacture micronized benzoyl peroxide (BP-M) first provide benzoyl peroxide as a feedstock to be used thereafter for the manufacture of benzoyl peroxide in a fatty substance. In the present invention this feedstock first is produced by a variety of ways as seen by the processes disclosed in U.S. Pat. No. 4,387,701, U.S. Pat. No. 4,497,794, U.S. Pat. No. 4,692,329, and U.S. Pat. No. 6,013,637. However, the resultant product size distribution of the benzoyl peroxide and the fatty material in which it is dispersed, results in a particle size distribution of benzoyl peroxide in related drug product of 35 microns or greater. This is consistent with benzoyl peroxide that has merely been de-agglomerated, rather than having been subjected to a process resulting in significant reduction of the primary particle size. De-agglomerated benzoyl peroxide can be used directly in the process to manufacture benzoyl peroxide in a fatty substance having a melting temperature of about 25° C. to about 45° C. Various de-aglommerization and/or micronizing techniques may alternatively be employed in achieving the micro-particle sizes that also comprise the present invention. U.S. Pat. No. 4,387,701, U.S. Pat. No. 4,497,794, U.S. Pat. No. 4,692,329, and U.S. Pat. No. 6,013,637 do not describe use of benzoyl peroxide for dispersion within a fatty substance to protect the benzoyl peroxide from interacting adversely with other ingredients of the pharmaceutical or cosmetic products in which it is used.

Canadian Patent No. 579,553 (Jul. 14, 1959) describes the preparation of finely divided benzoyl peroxide, at least 95 percent of whose particles have an average particle diameter of less than 12 microns and essentially 100 percent have an average particle diameter (d₁₀₀) below 20 microns. Processing is as a slurry or suspension in an organopolysiloxane fluid through a three-roller paint mill with the end-use of the micronized benzoyl peroxide being as a vulcanizing agent in silicone rubber manufacture. The requirement for the use of organopolysiloxane fluid as the liquid component of the slurry makes this option incompatible with therapeutic formulations for acne or other condition treatment and will interfere in the preparation of benzoyl peroxide in a fatty substance having a melting temperature of about 25° C. to about 45° C.

United States Patent Application No. 2004/0101566 (May 27, 2004) describes the preparation of nanoparticulate benzoyl peroxide with an effective average particle size (d₅₀) of less than about 2 microns. Specific options for the micronization of benzoyl peroxide to the desired particle size include milling, grinding, wet grinding, homogenizing and precipitation/crystallization. The descriptions appear centered around milling (specifically, grinding, wet grinding, and homogenizing). The processes described in this patent application may be used to prepare benzoyl peroxide for dispersion within fatty substances according to the present invention, although patent application 2004/0101566 does not describe this.

U.S. Pat. No. 4,401,835 (Aug. 30, 1983) describes the preparation of microparticulate benzoyl peroxide with a particle size of less than 10 microns by precipitation from a solvent/antisolvent system in the presence of a dispersant in comparison to material that had been prepared by milling in a roller mill to a particle size (d₁₀₀) of less than 250 microns as reported previously in the literature. This method necessitates the use of additional solvents/antisolvents, and the product must then be separated from the mother liquor and washed prior to use in the drug product formulation process. However, the benzoyl peroxide used to prepare the compositions of the present invention may be exposed to these processes to achieve a smaller particle size distribution. Thus the benzoyl peroxide produced in this way could be used for dispersion within the fatty substances of the present invention, although U.S. Pat. No. 4,401,834 does not describe this.

U.S. Pat. No. 7,820,186 (Oct. 26, 2010), United States Patent Application Nos. 2011/0003894 (Jan. 6, 2011), 2010/0160439 (Jun. 24, 2010), 2010/0029762 (Feb. 4, 2010), 2008/0181963 (Jul. 31, 2008), and World Patent Application No. 2008/006848 (Jan. 17, 2008) describe formulations for the treatment of acne incorporating microparticulate benzoyl peroxide having a particle size distribution characterized by a d₉₀ of less than 25 microns and treatment regimens for the use of said formulations. No mention is made of how this particle size is obtained, but the benzoyl peroxide appears to be pre-micronized. Poloxamer 124 is mentioned as a preferred dispersing/wetting agent. Propylene glycol is mentioned as a preferred penetrating agent. The benzoyl peroxide used to prepare the compositions of the present invention may be exposed to these processes to achieve a smaller particle size distribution. Thus, the benzoyl peroxide produced in this way could be used for dispersion within the fatty substances of the present invention, although the processes described in these patents do not describe such use of the benzoyl peroxide particles.

World Patent Application No. 2010/047784 (Apr. 29, 2010) (a.k.a. US 2010/099733) describes the preparation of micronized benzoyl peroxide as a suspension in water in the presence of a polyol, a polyol ether, or a low-carbon organic alcohol. Also discussed is a method for wetting a suspension of benzoyl peroxide in general, regardless of particle size, by a polyol, a polyol ether, or a low-carbon organic alcohol. The definition of micronized benzoyl peroxide within the patent is benzoyl peroxide whose particle size distribution is characterized by an average particle size (d₅₀) of greater than 150 microns. The benzoyl peroxide used to prepare the compositions of the present invention may be exposed to these processes to achieve a smaller particle size distribution. Thus, the benzoyl peroxide produced in this way could be used for dispersion within the fatty substances of the present invention, although this use is not described in patent application 2010/047784.

U.S. Pat. No. 6,117,843 (Sep. 12, 2000) describes a formulation for acne treatment containing microparticle benzoyl peroxide having an average particle size (d₅₀) of less than 35 microns. It does not claim or describe methods for benzoyl peroxide particle size reduction, but rather refers to the methods covered by the following patents: U.S. Pat. No. 3,535,422, U.S. Pat. No. 4,056,611, U.S. Pat. No. 4,387,107, and U.S. Pat. No. 4,923,900, also referenced herein. The compositions of the present invention may be exposed to these processes to achieve a smaller particle size distribution. Thus, the benzoyl peroxide produced in this way could be used for dispersion within the fatty substances of the present invention, although this use is not described in the aforementioned patents. U.S. Pat. No. 4,387,107 (Jun. 7, 1983), U.S. Pat. No. 4,497,794 (Feb. 5, 1985), U.S. Pat. No. 4,692,329 (Sep. 8, 1987), U.S. Pat. No. 6,013,637 (Jan. 11, 2000), describe therapeutic formulations employing pre-micronized benzoyl peroxide with a particle size distribution (d₁₀₀) of less than 150 microns and a mean average particle size distribution (d₅₀) of less than 35 microns. The use of non-micronized benzoyl peroxide is also described, with de-agglomeration being accomplished via milling of the formulated product. The benzoyl peroxide used to prepare the compositions of the present invention may be prepared by these processes to achieve a smaller particle size distribution. Thus, the benzoyl peroxide produced in this way could be used for dispersion within the fatty substances of the present invention, although this use is not described in the aforementioned patents.

U.S. Pat. No. 4,056,611 (Nov. 1, 1977), describes a formulation containing microparticle benzoyl peroxide with a particle size characterized by a d₁₀₀ of less than 100 microns, which can be accomplished by either milling the benzoyl peroxide prior to formulation, or milling the formulation mixture during the formulation process. No description of the milling method is supplied or claimed within this patent. The benzoyl peroxide used to prepare the compositions of the present invention may be prepared by these processes to achieve a smaller particle size distribution. Thus, the benzoyl peroxide produced in this way could be used for dispersion within the fatty substances of the present invention, although this use is not described in U.S. Pat. No. 4,056,611.

U.S. Pat. No. 3,535,422 (Oct. 20, 1970) describes a formulation containing microparticulate benzoyl peroxide with a particle size characterized by a d90 of less than 250 microns, which can be accomplished by either milling the benzoyl peroxide prior to formulation, or milling the product mixture during formulation. The benzoyl peroxide used to prepare the compositions of the present invention may be prepared by these processes to achieve a smaller particle size distribution. Thus, the benzoyl peroxide produced in this way could be used for dispersion within the fatty substances of the present invention, although this use is not described in U.S. Pat. No. 3,535,422.

U.S. Pat. Nos. 6,159,442 and 6,221,332 and United States Patent Application No. 2009/0269250 disclose equipment useful in the application of microfluidizer technology developed and marketed by Microfluidics International Corporation, incorporated herein by reference. Use of Microfluidics technology for the production of microparticulate benzoyl peroxide is neither reported nor disclosed in the patent literature or the literature in general. The benzoyl peroxide used to prepare the compositions of the present invention may be exposed to these processes to achieve a smaller particle size distribution. Thus, this technology can be used to prepare micronized benzoyl peroxide that can then be used in the manufacture of benzoyl peroxide in a fatty substance having a melting temperature of about 25° C. to about 45° C.

WO 2009/158687A1 relates to microemulsions having particle sizes of about 10 nanometers (0.010 microns) to 300 nanometers (0.3 microns), much smaller than the primary distribution (d₉₀) of microparticle sizes of the present invention.

The use of high shear mixers (rotor/stator mixers such as those manufactured by Silverson and Admix) for particle size reduction of benzoyl peroxide (in slurries of the type described in the examples below) has demonstrated that de-agglomeration is possible using these techniques. Thus, the benzoyl peroxide produced in this way can be used for dispersion within the fatty substances of the present invention.

The use of media mills, such as those supplied by NETZSCH Premier Technologies, can be used to micronize benzoyl peroxide to the particle size distribution levels described above. Thus, the benzoyl peroxide produced in this way can be used for dispersion within the fatty substances of the present invention.

The use of microfluidizing devices such as those made by MicroFluidics International Corporation and described in U.S. Pat. Nos. 6,159,442 and 6,221,332 and US patent application 2009/0269250 can also be used to reduce the particle size of commercially manufactured benzoyl peroxide to achieve the particle size distribution levels described above. These US patents do not however describe use of Microfluidics technologies for production of microparticulate benzoyl peroxide, nor for use of microparticulate benzoyl peroxide as a dispersion within a fatty substance according to the subject of the present invention.

The benzoyl peroxide in a fatty substance having a melting temperature of about 25° C. to about 45° C. resulting from the present invention is useful in the formulation of anti-acne compositions and other skin care therapies.

### SUMMARY

The compositions of the present invention comprise a first active ingredient within a fatty substance having a melting temperature of from about 25° C. to about 45° C. so that the first active ingredient is substantially contained within the fatty substance and prevents or mitigates interaction of the first active ingredient with other ingredients. In a preferred embodiment, the first active ingredient is benzoyl peroxide. The composition of the present invention's fatty substance is a solid at room temperature and melts or softens when applied to the skin of a subject.

The benzoyl peroxide within the fatty substance of the present invention is achieved by being substantially contained, entrapped, entailed, encapsulated (all meaning contained and interchangeable with one another), within the fatty substance, thereby segregating and protecting it from any other coformulated active pharmaceutical or cosmetic ingredients that may be utilized in the product formulation utilizing the benzoyl peroxide within the fatty substance.

Other active pharmaceutical and/or cosmetic ingredients can also be dispersed or dissolved within the fatty substance, separately in different particles of the fatty substance or together with the benzoyl peroxide in the fatty substance. Alternatively, the benzoyl peroxide can be the only pharmaceutical active ingredient within a fatty substance used in the resulting drug product formulations. Alternatively, other pharmaceutical and/or cosmetic ingredients that otherwise interact adversely with one another can also be dispersed or dissolved within fatty substances to segregate them from one another and to protect them from one another within pharmaceutical or cosmetic product formulations until the formulated product is applied to the skin allowing the ingredient or ingredients within the fatty substance to be released onto or into the skin by softening or melting of the fatty substance.

The dispersion of pharmaceutical active ingredient or ingredients or cosmetic ingredient(s) within a fatty substance can be used not only to protect these ingredients from one another but can also be used to protect these ingredients from other agents such as the water used in aqueous based pharmaceutical or cosmetic product formulations, or to protect these ingredients from aerial oxidation by the oxygen present in the air.

The dispersion of pharmaceutical active ingredient or ingredients within a fatty substance can be used not only to protect these ingredients from one another but can also be used to protect users of such pharmaceutical products from exposure to those active ingredients other than at the target site of the skin where these pharmaceutical products are applied at the time the pharmaceutical products are applied to the target area of the skin for treatment.

Once applied to the skin, the fatty substance softens and/or melts (hereinafter together referred to as “melts”), and the benzoyl peroxide and/or any other active ingredients are released or diffuse onto or into the skin or underlying tissues.

The method for preparing the benzoyl peroxide compositions comprises: (a) dispersing a fatty substance having a melting temperature of from about 25° C. to about 45° C. into droplets in an aqueous suspension comprising particles of benzoyl peroxide at a temperature of at least the melting temperature of the fatty substance in the presence of an emulsifying agent to provide a dispersion comprising droplets of the fatty substance having benzoyl peroxide therein; and (b) cooling the dispersion to about room temperature or colder, specifically to a temperature below the melting temperature of the fatty substance, to thereby solidify the fatty substance; thereafter, optionally isolating the solidified fatty substance particles containing the benzoyl peroxide by filtration and washing or by decantation and washing or by other means such as centrifugation.

In another aspect, the method for preparing the compositions containing benzoyl peroxide comprises: (a) suspending a composition comprising benzoyl peroxide and a fatty substance having a melting temperature of from about 25° C. to about 45° C., at a temperature of at least the melting temperature of the fatty substance to provide a suspension comprising the fatty substance and benzoyl peroxide; (b) forming droplets from the suspension; and (c) cooling and thereby solidifying the droplets to provide particles comprising benzoyl peroxide within the fatty substance followed by optionally isolating the solidified fatty substance particles containing the benzoyl peroxide by filtration and washing or by decantation and washing or by other means such as centrifugation.

A further method for preparing the benzoyl peroxide compositions of the present invention is by dispersing the benzoyl peroxide into the melted fatty substance, such as melted cocoa butter, and thereafter mixing the resulting mixture into or with an aqueous medium that optionally comprises a surfactant, such as Tween 20, 80 or the like. Preferably, the benzoyl peroxide and fatty substance, such as cocoa butter, slurry is added into the surfactant [Tween]/water mixture. The fatty substance containing the benzoyl peroxide (and/or other active ingredient or ingredients) is cooled before, during, or after the addition of the benzoyl peroxide slurry to the water-continuous phase (with or without Tween in the water phase).

A further method for preparing the compositions of the present invention is as follows: (a) disperse benzoyl peroxide and/or other pharmaceutically and/or cosmetically active ingredients into the melted fatty substance, such as cocoa butter; (b) followed by dispersing the molten mixture into a mixture of water and surfactant with vigorous agitation to achieve fine droplets of the molten fatty substance within the aqueous mixture; (c) thereafter, cooling this resultant mixture below the melting point of the fatty substance, in order to solidify the fatty substance with the benzoyl peroxide and/or the other pharmaceutical active ingredients contained therein; (d) isolating by filtration or decantation or centrifugation or other means; and (e) then washing and drying the product. The resultant product contains just benzoyl peroxide within the fatty substance particles or a mixture of benzoyl peroxide and other pharmaceutical and/or cosmetic active ingredients. Alternatively, the product can contain only the other pharmaceutical or cosmetic active ingredients. The order of addition of the various components can be altered.

The solid particles of the fatty substance containing benzoyl peroxide and/or other pharmaceutical and/or cosmetic active ingredients are then formulated into products used for treating acne or other skin diseases by mixture with excipients and other product ingredients in order to achieve a formulation that is packaged, stored, distributed and sold for application to the skin to treat diseases of the skin and/or the underlying tissue below the skin.

The method for delivering the pharmaceutical or cosmetic compositions comprising benzoyl peroxide and/or other active ingredient or ingredients to the skin of a subject, comprises: applying products containing a composition comprising benzoyl peroxide and/or other active ingredients within a fatty substance having a melting temperature of from about 25° C. to about 45° C. to the skin of a subject, including mammals (e.g. humans, domestic animals, and other animals).

The present invention also relates to a process for dispersing micronized benzoyl peroxide (with a primary particle size distribution characterized by a d₉₀ from about 0.1 to 150 microns but preferably with d₉₀ in the range of about 10 to 15 microns) and/or other pharmaceutical active ingredient or ingredients, into a fatty substance having a melting temperature of about 25° C. to about 45° C. where the fatty substance particle size distribution can also be controlled to achieve d₉₀ from 0.1 to 200 microns, more preferably a d₉₀ from 10 to 50 microns, and most preferably a d₉₀ from 15 to 25 microns.

The present invention furthermore relates to the formulations of micronized benzoyl peroxide and product compositions resulting from such process.

The present invention also relates to methods for treating skin diseases such as acne, bacterial skin diseases, yeast skin diseases, fungal skin diseases, and/or skin diseases caused by virus infection, by administering the compositions of the present invention to the surface of the skin and/or to treat the underlying tissue by transdermal effect through the surface of the skin or by subcutaneous application. Furthermore, diseases of the skin or the tissue underlying the skin such as skin cancer or other cancers may be treated by the compositions of the present invention wherein the fatty substance containing the active ingredient or active ingredients acts as a reservoir slowly releasing the active ingredient or ingredients from the fatty substance onto the skin or through the skin to treat the underlying tissue.

Compositions of active ingredient or ingredients present in fatty substance present in pharmaceutical product formulations may be injected under the surface of the skin so that the fatty substance acts as a subcutaneous reservoir slowly releasing the active ingredient or ingredients under the surface of the skin to treat the underlying tissue. The active ingredient or ingredients may be dispersed within particles of the fatty substance that are in turn formulated with other excipients within an aqueous based pharmaceutical product formulation, or they may be dispersed within the fatty substance which is warmed to melt the fatty substance before injecting it under the surface of the skin.

Once applied onto or under the skin, the fatty substance softens and/or melts (hereinafter together referred to as “melts”), and the benzoyl peroxide and/or any other active ingredients are released or diffuse onto or into the skin or underlying tissues.

### BRIEF DESCRIPTION OF THE DRAWING

FIG. 1 illustrates the embodiments of the present invention. The rectangle illustrates the aqueous-based formulation matrix wherein particles of the fatty substance containing various active ingredients within the fatty substance or outside of the fatty substance are located. The oval shapes represent the fatty substance, such as cocoa butter. The triangles represent the first active ingredient, such as benzoyl peroxide, contained within the fatty substance and thereby protected from other ingredients. The diamonds represent other active ingredients, either within the fatty substance or outside of the fatty substance, but in both situations, the first ingredient is substantially protected from adversely chemically interacting with any other active ingredients by virtue of the segregation or separation effect afforded by the fatty substance.

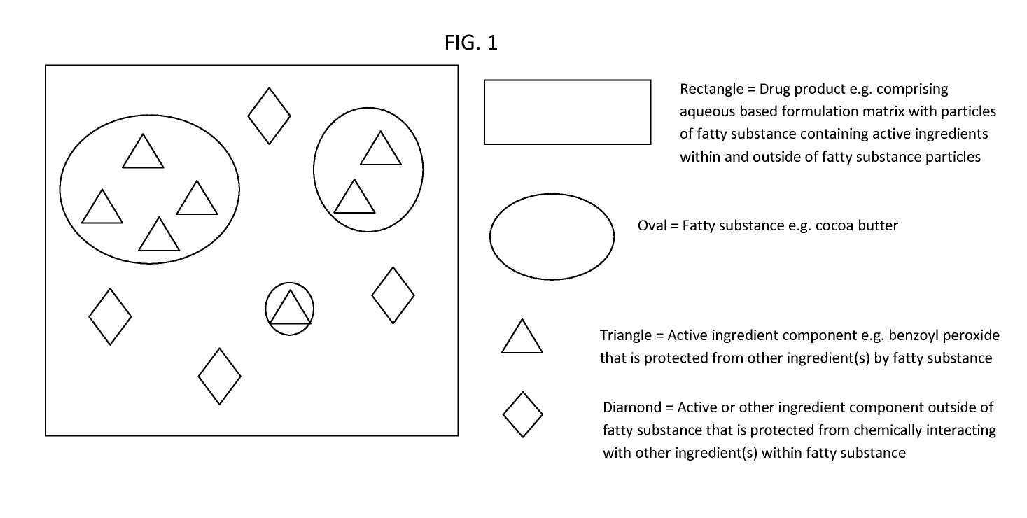

### DETAILED DESCRIPTION

The present invention provides for a low cost, easily manufactured and non-toxic means to prepare compositions comprising benzoyl peroxide, either alone or in combination with other active ingredients suitable for pharmaceutical, cosmetic skin care, or animal health care products such that the active ingredients are substantially prevented from reacting with one another in the composition while still allowing for the active ingredients to have an effect after being applied to the skin, or transdermally through the skin or subcutaneously under the skin. The compositions comprise benzoyl peroxide and any other active ingredients that are useful in a variety of such formulations suitable for administration topically, transdermally and/or subcutaneously. The benzoyl peroxide compositions are formulated into pharmaceutical or cosmetic formulations and delivered to the skin of a subject, preferably a human.

The compositions comprise benzoyl peroxide and/or other active ingredient or ingredients within a fatty substance having a melting point of from about 25° C. to about 45° C., which is around the mammalian, preferably human, body temperature of about 37° C. A substance that is “within” the fatty substance means that the substance is dissolved, dispersed, suspended, encapsulated, segregated, embedded, contained, or entrained (all used interchangingly here) in the fatty substance. The benzoyl peroxide is present within the fatty substance in amounts ranging from about 10% to about 90%, about 25% to about 90%, about 50% to about 90%, or about 60% to about 90% by weight of the fatty substance. Additional pharmaceutical and/or cosmetic ingredients are also optionally present within the fatty substance in amounts ranging from between about 0.1% and about 70% according to the amount needed for therapeutic dosing. For example the fatty substance in the form of small particles may contain 70% by weight of benzoyl peroxide and 0.25% by weight of a retinoid and may then be formulated with 10 parts by weight of aqueous based drug product formulation components to produce a drug product containing 7% by weight of benzoyl peroxide and 0.025% by weight of retinoid.

The benzoyl peroxide in fatty substance compositions optionally comprise other active pharmaceutical and/or cosmetic ingredients present in the fatty substance or present in formulated pharmaceutical and/or cosmetic products but outside of the fatty substance, such that the benzoyl peroxide is substantially prevented from coming into contact with and reacting with the other active pharmaceutical or cosmetic ingredients during formulation and storage of the cosmetic and/or pharmaceutical products since the benzoyl peroxide is substantially prevented from interaction with the other active pharmaceutical or cosmetic ingredients or ingredients by the fatty substance in which the benzoyl peroxide is substantially contained. The fatty substance particles containing active ingredient or ingredients may be dispersed with or without other excipients within an aqueous base to prepare pharmaceutical product or cosmetic product formulations. The aqueous based formulation mixture is also referred to as the formulation matrix. A pharmaceutical and/or cosmetic ingredient or ingredients that are present in the formulation matrix may be absorbed onto the fatty substance particles or substantially segregated from the fatty substance and/or the benzoyl peroxide altogether. Optionally, any additional pharmaceutical and/or cosmetic ingredients are freely present in the formulated product matrix (outside or separate from the benzoyl peroxide present in the fatty substance particles) in amounts ranging from about 0.01% to about 20%, or about 1% to about 15%, by weight of the formulated drug or cosmetic product using compositions based on the present invention. Such a co-formulation can be prepared as a pharmaceutical or cosmetic formulation as described below. In other examples, additional pharmaceutical or cosmetic ingredients are present within or on the fatty substance, but preferably segregated from the benzoyl peroxide, e.g., present in separate fatty substance particles or are kept separate from the benzoyl peroxide by themselves being immobilized within fatty substance particles separate from the fatty substance particles in which the benzoyl peroxide is substantially immobilized. Both the benzoyl peroxide and the other active ingredient or ingredients also may be immobilized within the same fatty substance particles, relying on the solidification of the fatty substance on cooling below the melting temperature of the fatty substance during preparation of the fatty substance containing the benzoyl peroxide and the other active ingredient or ingredients. The particles of fatty substance containing benzoyl peroxide or other active ingredient(s) are then combined with other active ingredient(s) and/or other ingredients during formulation of final products suitable for application to the skin.

The compositions comprising benzoyl peroxide (or other reactive or sensitive ingredients) present in fatty substance that is formulated into drug or cosmetic products optionally include other pharmaceutical or cosmetic active ingredients (either free within the formulation matrix but outside of the fatty substance, or within, or on the fatty substance) suitable for the treatment of skin and other medical indications such as acne, warts such as plantar warts, herpes simplex virus infections such as cold sores or other herpes infections, psoriasis, seborrhea, rosacea, vitiligo, onychomycosis, athlete's foot, bacterial skin infections, skin yeast infections, skin fungal infections, and cancers such as melanoma or breast cancer. Examples of other suitable pharmaceutical and/or cosmetic ingredients include antibiotics, retinoids, fungicides, vitamins, steroids, antivirals, anticancer agents and other ingredients used for the treatment of skin or underlying tissue. Specific examples of active ingredients, in addition to, or instead of, benzoyl peroxide, that are optionally present in the composition or formulation, either individually or in combination, include compounds for treating warts such as bleomycin, 2,4-dinitrochlorobenzene, fluorouracil, salicyclic acid, silver nitrate, zinc sulfate, zinc oxide, cantharidin, podophyllin, or imiquimod, antibiotics such as clindamycin and its salts such as clindamycin hydrochloride and clindamycin phosphate, erythromycin, tetracycline, dicloxacilin, doxycycline, minocycline, bacitracin, chlortetracycline, neomycin, mupirocin, polymyxin B, cuprimyxin, furazolidone, gentamycin, lincomycin, cephalosporins, betalactam antibiotics, and their salts such as lincomycin hydrochloride and other antibiotics; retinoids such as tazarotene, vitamin A, retinoic acid, tretinoin, isoretinoin, adapalene, retinol, aciretin, bexarotene and other retinoids; oxybutynin; vitamin D, vitamin C, vitamin B, vitamin E and other vitamins; sulfur; glucocorticosteroids, corticosteroids, triamcinolone, triamcinolone acetonide, betamethasone, betamethasone 17-valerate, betamethasone dipropionate, halcinonide, isoflupredone acetate, flumethasone, fluocinonide, mometasone, fluticasone, fluticasone propionate, prednisolone, beclomet(h)asone, hydrocortisone, and other steroids; cyproterone, drospirenone, estrogen, progestogen and other hormones; tacrolimus, pimecrolimus, ursolic acid, betulinic acid, moronic acid, oleanolic acid, acyclovir, valaciclovir, famciclovir, penciclovir, docosanol, perillyl alcohol, cyclophosphamide, methotrexate, doxorubicin, paclitaxel, docetaxel, epirubicin, vemurafenib, gefitinib, anastrazole, letrozole, aromasin, tamoxifen, dacarbazine and other anti-cancer agents; antiviral agents such as acyclovir, uclacyclovir, famiciclovir, penciclovir; immunosuppressive agents such as tacrolimus and pimeorolimus; or anti-inflammatory agents; antifungal agents such as itraconazole, fluconazole, voriconazole, ketoconazole, miconazole, miconazole nitrate, clotrimazole, sulconazole nitrate, terbinafine, econazole nitrate, tioconazole, itraconazole, posaconazole griseofulvin, nystatin, amphotericin B, neticonazole, butenafine, lanoconazole, terconazole, butoconazole, bifonazole, isoconazole, fezatione, tolnaftate, flucytosine, clioquinal, ticlatone, haloprogin, ciclopirox, natamycin and other antifungal agents; tea tree oil, selenium sulfide, acetyl salicylic acid, amorolfine, anthralin, nizoral, coal tar, resorcinol, glycolic acid, witch hazel, and alpha hydroxyl acids among other pharmaceutical ingredients useful for skin treatment.

The fatty substance of the composition includes a variety of hydrophobic or lipophilic materials having a melting temperature of from about 25° C. to about 45° C. The fatty substance preferably has a melting temperature of from about 30° C. to about 38° C. Generally, the fatty substance comprises a variety of glycerol fatty acid esters (tri-esters and mixtures of esters), as well as natural fats derived from animal, vegetable and/or mineral origins and/or synthetic fats that are partially or fully dehydrogenated, as long as they fulfill the melting temperature parameters of the present invention. Some specific examples include individual components or mixtures chosen from olive oil, corn oil, castor oil, cottonseed oil, wheat germ oil, cacao butter, hydrogenated oils, etc.; hydrocarbons, e.g., squalane, petrolatum, solid paraffin, liquid paraffin, etc.; and waxes, e.g., jojoba oil, carnauba wax, bees wax, lanolin, etc., and other oily or fatty substances. Exemplary fatty substances comprise a variety of saturated and/or unsaturated fats, such as trilaurin, stearic acid, palmitic acid, capric acid, myristic acid, arachidic acid, lauric acid, oleic acid, palmitoleic acid, linoleic acid, linolenic acid, lauric acid, as well as esters, such as triglycerides, thereof. Further, tallow fat, cochineal fat, dog grease, duck grease, palm oil and whale blubber can be used. A listing of such fats can be found in Spons' Encyclopedia of the Industrial Arts, Manufactures and Commercial Products, pages 1351-1432 (1879), incorporated herein by reference.

Commercial products of these components include Witepsol (manufactured by Dynamit Nobel), Pharmasol (manufactured by Nippon Oil and Fats Co.), Isocacao (manufactured by Kao Corp.), SB (manufactured by Taiyo Oil and Fats Co.), Novata (manufactured by Henkel), Suppocire (manufactured by Gattefosse Co.), and the like. Polyethylene glycol, e.g., macrogol, cetomacrogol, etc., as well as derivatives thereof, e.g., cetomacrogol, are given as examples of other synthetic products.

In some examples, the fatty substance comprises a butter or fat derived from a plant or animal source, such as lard, butter, palm oil, or vegetable oils. Specific examples include kapok, esparto, cocoa butter, shea butter, mango butter, kokum butter, and mixtures thereof. Cocoa butter, for example, comprises a variety of fats such as saturated fats, stearic acid, palmitic acid, capric acid, myristic acid, arachidic acid, lauric acid, unsaturated fats, monounsaturated fats, oleic acid, palmitoleic acid, polyunsaturated fats, linoleic acid, and linolenic acid, including a variety of esters and triglycerides thereof. Cocoa butter is a commodity product and is readily available in high quality for use in pharmaceutical or cosmetic formulations and has a melting range ideally suited to skin temperature.

The desirable melting point of the formulations of the present invention can be obtained by combining fatty substances. For example, in order to decrease the melting point, a plasticizer can be added, such as glyceryl monostearate, myristyl alcohol, polysorbate 80, propylene glycol, or a combination thereof. In order to increase the melting point, a hardener can be added, such as beeswax, cetyl alcohol, stearic acid, stearyl alcohol, aluminum monostearate, aluminum distearate, aluminum tristearate, bentonite, magnesium stearate, colloidal silicon dioxide and combinations thereof.

A carrier for the present invention used in pharmaceutical and/or cosmetic products utilizing the fatty substance to segregate and protect mutually incompatible ingredients may comprise any ingredient suitable for use in a pharmaceutical formulation. For example, the carrier may include a cellulose or may be one or more ingredients selected from the group consisting of glycerol esters of saturated fatty acids or one or more polyglycolysed glycerides, cocoa butter, theobroma or the like, one or more high molecular weight polyethylene glycol, one or more polyoxyethylene, lanolin and derivatives thereof, and one or more fatty acids, fatty alcohols, fatty acid esters (including, for example, caprylic acid, caprylic triglyceride or the like). These ingredients can be optionally mixed with one or more organic oils (including, for example hydrogenated vegetable oils) or the like to achieve the desired component's melting point.

Further examples of fatty substances useful in the present invention that have a melting point of about 25° C. to about 45° C., more specifically of about 30° C. to about 38° C. include decanoic acid, undecanoic acid, erucic acid, tetradecanol, tridecanol, lauryl alcohol, heneicosane, nonadecane, octadecane, eicosane, elemi resin, levulinic acid, palm oil, coconut oil, dimethyl sebacate, adipic acid, monoethyl ester, polyethylene glycol (Mn660 and 950-1050), Brij® S10, Brij® 98, Brij® C10, Brij® L23, and Brij® 52, diethylene glycol monotetradecyl ether, diethylene glycol monotetradecyl ether, heptaethylene glycol monododecyl ether, palmitate esters and/or stearate esters, polycaprolactone-block-polytetrahydro-furan-block (Mn=1000, 2000 and 2900), poly[di(ethylene glycol) adipate] (M=500), and poly[trimethylolpropane/dipropylene glycol-alt-adipic acid/phthalic anhydride] polyol (Mn=500).

Droplets of the fatty substance having benzoyl peroxide and any other ingredients therein can be prepared by dispersing or suspending benzoyl peroxide (and other optional ingredients) in water or an aqueous solution or dispersion in the presence of an emulsifying agent, such as Tween 20 (polyoxyethylene (20) sorbitan monolaurate), Tween 80 (polyoxyethylene (80) sorbitan monolaurate), polysorbate detergent, tetralkylammonium salts, such as benzalkonium chloride, water and oil co-miscible solvents such as alcohols, polyvinyl pyrrolidone, mustard extract, or other non-toxic emulsifiers. The emulsifying agent facilitates dispersion of the fatty substance into the aqueous continuous phase. Surfactants such as Tween reagents are helpful for wetting and dispersing the benzoyl peroxide and any other ingredients present and facilitating dispersion of the benzoyl peroxide and any other ingredients into the fatty substance. Alternatively, the benzoyl peroxide and fatty substance (cocoa butter) mixture or slurry are mixed together and then added to a surfactant [such as the above-listed Tweens] and water mixture, with stirring.

The fatty substance is added to the suspension or dispersion of benzoyl peroxide and any other ingredients with stirring. The resulting mixture is then heated at a temperature of at least the melting point of the fatty substance while mixing such that droplets of the fatty substance are formed and dispersed in the aqueous continuous phase of the mixture. Alternatively, the mixture is heated prior to the addition of the fatty substance. The temperature at which the mixture is heated is preferably at least the melting temperature of the fatty substance and no greater than 15° C., 10° C., or 5° C. greater than the melting temperature of the fatty substance. For example, the mixture is heated at a temperature of from about 28° C. to about 50° C., or from about 33° C. to about 45° C., to melt the fatty substance. The fatty substance preferably has a good propensity for physical association with the benzoyl peroxide such that the benzoyl peroxide is dispersed into the fatty substance particles amidst the water, emulsifying agent, and any other ingredients.

Once the benzoyl peroxide is dispersed into the fatty substance (which is itself dispersed into droplets), the temperature of the mixture is cooled to below the melting point of the fatty substance such that the fatty substance solidifies with the benzoyl peroxide therein. Once solidified, the solid is isolated and washed free of the aqueous phase by filtration or by decantation and washing followed by further decantation and/or by filtration and washing or by other means, provided that the temperature during isolation is maintained below the melting temperature of the fatty substance. The isolated particles are used as a wet formulation or dried by vacuum drying or fluid bed drying, while maintaining the temperature below the melting point of the fatty substance.

The rate and extent of mixing as well as the method of agitation and the ratio of the water to emulsifying agent to fatty substance is adjusted to facilitate larger or smaller droplet sizes, depending on the desired droplet size or solid particle size range. Benzoyl peroxide particles of varying sizes may also be used in conjunction with varying fatty substance droplet sizes in order to obtain variation in the overall surface area of both the fatty substance particles obtained after cooling, as well as variation in the overall surface area of the benzoyl peroxide that is within the fatty substance. The increased surface area (from smaller particles) has the effect of making the benzoyl peroxide more quickly available for release at the skin surface or for absorption through the skin to affect the underlying tissue.

Other pharmaceutical active ingredients or cosmetic ingredients may similarly be dispersed into fatty substance in lieu of, or in addition to, benzoyl peroxide.

The pharmaceutical active ingredient(s) or cosmetic ingredients and the fatty substance are mixed in such a way as to disperse the pharmaceutical active ingredient(s) into the molten fatty substance. The molten fatty substance is dispersed into small droplets within a continuous liquid medium such as water, aided by a surfactant to stabilize the small droplets of the fatty substance. The temperature of the mixture is then reduced below the melting temperature of the fatty substance such that the fatty substance droplets containing the active pharmaceutical ingredient(s) or cosmetic ingredient(s) are solidified. The resultant particle size distribution for the fatty substance is characterized by a d₉₀ of 0.1 to 200 microns, more preferably by a d₉₀ from 5 to 50 microns, and most preferably by a d₉₀ from 15 to 30 microns.

A method for preparing the composition of the present invention comprises dispersing a fatty substance such as lard, butter, palm oil, cocoa butter or other materials having melting temperature between about 25° C. and about 45° C. into droplets in an aqueous suspension at a temperature of at least the melting temperature of the fatty substance along with an emulsifying agent, such as Tween 20 (polyoxyethylene (20) sorbitan monolaurate), Tween 80 (polyoxyethylene (80) sorbitan monolaurate), polysorbate detergent, tetralkylammonium salts, such as benzalkonium chloride and/or with oil co-miscible cosolvents such as alcohols, polyvinyl pyrrolidone, mustard extract, or other non-toxic emulsifiers. To this mixture is added the active pharmaceutical ingredient which can be microparticulate benzoyl peroxide having particle size distribution with d₉₀ from 0.1 to 150 microns, more preferably by a d₉₀ from 5 to 25 microns, and most preferably by a d₉₀ from 10 to 15 microns, or other API, so as to provide a dispersion comprising droplets of the fatty substance having the API dispersed within the droplets of the fatty substance. The said suspension is subjected to high shear forces that produce fatty droplets that measure from 0.1 micron to 200 microns or smaller. The mixture is cooled to about room temperature or lower to solidify the fatty substance. Thereafter, the solidified fatty substance is isolated by filtration or other means to provide particles of fatty substance with particle size distribution of d₉₀ from 0.1 to 200 microns, more preferably by a d₉₀ from 10 to 50 microns, and most preferably by a d₉₀ from 15 to 25 microns, where the active pharmaceutical ingredient dispersed within these particles of fatty substance is benzoyl peroxide that may have been pre-micronized. That benzoyl peroxide has particle size distribution with d₉₀ from 0.1 to 150 microns, more preferably with d₉₀ from 5 to 25 microns, and most preferably with d₉₀ from 10 to 15 microns. More specific examples of fatty substance include kapok, esparto, cocoa butter, shea butter, mango butter, kokum butter, and mixtures thereof with melting temperature anywhere between about 25° C. and about 45° C. Cocoa butter, for example, comprises a variety of fats such as saturated fats, stearic acid, palmitic acid, capric acid, myristic acid, arachidic acid, lauric acid, unsaturated fats, monounsaturated fats, oleic acid, palmitoleic acid, polyunsaturated fats, linoleic acid, and linolenic acid, including a variety of esters and triglycerides having a melting temperature range of between around 30° C. and 38° C.

A further method for preparing the benzoyl peroxide in fatty substance compositions of the present invention is by dispersing the benzoyl peroxide (with or without premicronization) and/or other pharmaceutical active ingredient or ingredients or cosmetic ingredient(s) into the fatty substance above its melting temperature, and thereafter adding water and optional surfactant. Alternatively, add the dispersion of the pharmaceutical active ingredient in the melted fatty substance into water and optional surfactant, followed by subjecting said aqueous suspension that optionally includes a surfactant, such as Tween 20, 80 or the like to high shear forces that produce fatty droplets that measure from 0.1 micron to 200 microns. Preferably, the fatty substance, such as cocoa butter, is added into benzoyl peroxide slurry which contains the surfactant [Tween]/water mixture.

The device used to achieve fine particle size droplets of the melted fatty substance having a melting temperature of about 25° C. to about 45° C. mixed with benzoyl peroxide and/or other active pharmaceutical ingredient or cosmetic ingredient can be a microfluidizer (Microfluidics International Corporation), high shear mixer (Silverson and Admix) or any piece of equipment that is able to achieve fine droplets of the fatty substance within the aqueous or other medium in which it is being mixed. The active ingredient or ingredients is dispersed within the fatty substance, before the temperature is reduced to a temperature below the melting temperature of the fatty substance in order to freeze the fatty substance with the active ingredient or ingredients then frozen within the fatty substance particles. The resultant frozen composition is then isolated by decantation, filtration, centrifugation or other means before subsequently being formulated with other ingredients, such as excipients, into a water-based gel as a pharmaceutical product or cosmetic product. The particle size of the fatty substance particles produced in such a manner is characterized by particle size distribution d₉₀ of between 0.1 micron and 200 microns, preferably between d₉₀ from 5 to 50 microns.

Shear rates that can be obtained by rotor-stator mixers ranging from 500,000 to 700,000 sec-1, and/or other methods disclosed above, are necessary in order to reduce the primary particle size of the fatty substance to a d₉₀ of 0.1 to 200 microns, more preferably to a d₉₀ from 5 to 50 microns, and most preferably to a d₉₀ from 15 to 30 microns. Use of benzoyl peroxide in a fatty substance having a melting temperature of about 25° C. to about 45° C. characterized by the desired particle size distribution range described above in therapeutic applications for acne or other skin treatments is desirable due to a more homogeneous distribution of the benzoyl peroxide in a fatty substance having a melting temperature of about 25° C. to about 45° C. within the formulation, higher bioavailability due to increased surface area of the particles on a weight basis, prevention of locally high benzoyl peroxide concentrations on the skin which in turn result in skin irritation in the vicinity of said particles/agglomerates, and avoidance of large/agglomerated particles (outside of the particle sizes claimed herein) which can lead to a chunky appearance.

Preparation of microparticulate benzoyl peroxide in a fatty substance having a melting temperature of about 25° C. to about 45° C. at the more preferred particle size d₉₀ of 0.1 to 200 microns, more preferably by a d₉₀ from 5 to 50 microns, and most preferably by a d₉₀ from 15 to 30 microns is generally accomplished by the following process:

As a non-limiting general description of the process embodied herein, an aqueous slurry of benzoyl peroxide is prepared such that the benzoyl peroxide content is preferably from 1 to 75 wt % relative to the total mass of the slurry. This slurry also contains dispersant(s) at levels appropriate/required to maintain the benzoyl peroxide and benzoyl peroxide in a fatty substance having a melting temperature of about 25° C. to about 45° C. as a free flowing slurry that is easy to maintain in a fluid state without significant foaming or air entrainment under the level of agitation required for slurry maintenance. Into this slurry is added a fatty substance having a melting temperature from about 25° C. to about 45° C. This slurry is heated to a temperature which meets or safely exceeds the melting point of the fatty substance and is then processed in a rotor-stator mixer or microfluidizer. This suspension is preferably cooled by transferring the suspension to an ice-water bath. The solid benzoyl peroxide in a fatty substance having a melting temperature of about 25° C. to about 45° C. is collected by filtration and dried.

Optionally, additional pharmaceutical or cosmetic formulation ingredients are also present in the same or separate droplets of the fatty substance, which are then cooled to generate solid fatty substance particles. Alternatively, the other pharmaceutical or cosmetic active ingredients are dispersed in the fatty substance in a stepwise manner whereby the benzoyl peroxide active is dispersed into the fatty substance that is itself dispersed into droplets. Then, the mixture is cooled to below the melting point of the fatty substance, after which time the other pharmaceutical active ingredient(s) is added and diffuses into or onto the surface of the fatty substance while the benzoyl peroxide remains within the fatty substance particles. These materials are then suspended within aqueous or other media to prepare products suitable for therapeutic and/or cosmetic application to the skin.

A method for preparing compositions comprising a treatment for melanoma or other skin cancers involves adding an anticancer drug such as dacarbazine to the mixture of the fatty substance in either an aqueous system or as a melt where the temperature exceeds the melting temperature of the fatty substance. The resulting fatty substance can then be applied to the affected area either topically, transdermally or subcutaneously.

A method for preparing compositions comprising an ionizable pharmaceutical and/or cosmetic ingredient involves adding the ionizable ingredient (e.g., retinoid) to the mixture of the fatty substance and benzoyl peroxide (or the aqueous suspension of benzoyl peroxide), at a pH greater than the pKa of the ionizable substance. For example, retinoid acid, tretinoin, or isoretinoin, can be added at pH of 5 to 10, preferably at pH of 6 to 8. The ionizable ingredient is preferentially soluble in the aqueous phase of the mixture while the benzoyl peroxide is preferentially within the fatty substance.

The ionizable active ingredient is added either before or preferably after the benzoyl peroxide is dispersed within the fatty substance. After the addition of the ionizable ingredient, the pH of the mixture is reduced to below the pKa of the ionizable ingredient such that the ingredient is protonated and rendered preferentially soluble in the fatty substance rather than the aqueous phase. The protonated ionizable material is then absorbed into or onto the droplets of the fatty substance. The pH is preferably reduced below the pKa of the ionizable material after the temperature of the mixture is lowered below the melting point of the fatty substance such that the fatty substance is solidified, and the protonated ionizable material diffuses into or onto the surface of the fatty substance, such that the ionizable ingredient and the benzoyl peroxide are kept substantially separate from one another to mitigate unwanted reactions between the materials. The pH may also be reduced before the temperature is reduced. The target pH to encourage retinoic acid or tretinoin to be adsorbed through the surface of the fatty substance droplets or particles is, for example, between pH 1 and 4 and preferably between pH 2 and 3 (the pKa of tretinoin is 3.8).

Different pharmaceutical and cosmetic active ingredients which interact with benzoyl peroxide resulting in mutual degradation, such as those discussed above, e.g., combinations of vitamin A, tretinoin, tazarotene, or another retinoid, are ideally prepared in other particles of fatty substance, separate from the particles containing benzoyl peroxide or other active ingredients, or are incorporated into formulated pharmaceutical product(s) outside of fatty substance particles and separate from the benzoyl peroxide or other incompatible ingredient(s) present in fatty substance, before or after benzoyl peroxide is incorporated within separate particles or the same particles of the fatty substance. The separately prepared active compositions are then mixed together in pharmaceutical or cosmetic skincare product formulations, such that the otherwise incompatible materials (such as benzoyl peroxide and retinoids) do not come into contact (and do not degrade one another during formulation or storage of the formulated product) and are then released when applied to the skin of subject.

The various incompatible pharmaceutical or cosmetic active ingredients can also be combined within given fatty substance particles. Although not wanting to be limited by theory, it is believed that freezing of the fatty substance particles before isolation to keep the incompatible active ingredients separate from one another even though they are together within the fatty substance particles, renders them immobilized and unable to interact with one another due to the freezing of the fatty substance particles. The frozen fatty substance particles containing the ingredients then can be formulated into final products with other excipients. The same also applies to formulations using the fatty substance as a solid form which is melted for dosing to patients as a topical application or as a subcutaneous deposit beneath the skin. In this situation, the active ingredients are released slowly from the fatty substance but are immobilized from interacting with one another whilst the fatty substance is kept in a frozen state.

An example of this is to formulate fluorouracil cancer treatment agent with methotrexate cancer treatment agent within melted cocoa butter. The melted mixture is then cooled to solidify the cocoa butter in order to mitigate adverse interaction between the two active ingredients before the mixture is then warmed to allow it to be dispensed onto the surface of the skin or injected below the surface of the skin.

A further example is to use the fatty substance to segregate water sensitive active ingredient or ingredients from an aqueous-based pharmaceutical product formulation such that the water-sensitive active ingredient is dispersed within the fatty substance particles. These particles are then isolated and formulated into a pharmaceutical product by mixing with aqueous based pharmaceutical product formulation components. Again, not wanting to be limited by theory, it is believed that this process keeps the water-sensitive active ingredient separate from the aqueous-based formulation components to enhance stability until the resultant mixture is applied onto or under the skin. When this application is made, the moltent or softened fatty substance is then released with the active ingredient or ingredients being applied onto the skin or into the underlying tissue.

Air sensitive pharmaceutical or cosmetic product ingredients are similarly protected by dispersion into fatty substance melt or molten droplets, followed by cooling below the melting temperature of the fatty substance in order to allow isolation of the fatty substance particles containing the air sensitive ingredients. These are then formulated with other excipients or ingredient(s) into products whereby the fatty substance keeps the air sensitive ingredients segregated from exposure to air.

Another method for preparing the compositions involves suspending the benzoyl peroxide (and/or other ingredient or ingredients) in the melted fatty substance under mixing conditions, followed by forming droplets from the slurry. Small droplets may be made by spraying the slurry through a small nozzle to cause formation of droplets or by pumping the slurry through an orifice to cause it to produce a continuous stream which is broken up by a rotating blade or a series of blades or wires to convert the stream to short columns, which then coalesce into droplets. Droplets of the fatty substance containing the benzoyl peroxide are solidified by cooling, either through the use of a chilled air stream or by letting the droplets fall into a chilled bath containing a chilled heat transfer fluid such as chilled water. The solid particles comprising the solidified fatty substance containing benzoyl peroxide and/or other active ingredients are then isolated by filtration or decantation or other means.

Another method for preparing the benzoyl peroxide compositions of the present invention involves dispersing the benzoyl peroxide into the fatty substance, such as a melted cocoa butter, and thereafter mixing the resulting mixture with water (with or without a surfactant, such as Tween 20, 80 or the like). Preferably, the benzoyl peroxide and fatty substance, such as cocoa butter, slurry is added into the water with vigorous stirring at a temperature above the melting temperature of the fatty substance followed by cooling. Alternatively, the slurry is added to cooled water, so that fatty substance droplets solidify as they are formed or the mixture is cooled during the addition of the slurry to water.

Another method for preparing the benzoyl peroxide compositions of the present invention involves combining the benzoyl peroxide and a fatty substance, such as a melted cocoa butter, into a mixture of water (with or without a surfactant such as Tween 20, 80 or the like). The resultant slurry is vigorously mixed at a temperature above the melting temperature of the fatty substance, followed by cooling. Alternatively, the resulting slurry is added to cooled water at a temperature below the melting point of the fatty substance, for example between 0° C. and 20° C., in order for the fatty substance droplets to solidify as they are formed, or the resulting mixture is cooled below the melting point of the fatty substance, for example to a temperature between 0° C. and 20° C. during the addition of the slurry to the water.

The particle size and particle size distribution of the benzoyl peroxide and any other ingredient is controlled and tailored to the desired size of the final isolated particles. For example, if the droplets of fatty substance containing the benzoyl peroxide and any other ingredient are formed with diameters of up to 1 mm, then smaller particles of benzoyl peroxide in the range 100 microns or less may be used as the starting material to help achieve uniformity of distribution of the benzoyl peroxide throughout the fatty substance. Even smaller benzoyl peroxide particles or ingredient particles are useful to provide an encapsulated product having an average particle size of 50 microns, 30 microns, 10 microns, or even less.

The compositions comprising benzoyl peroxide and any other pharmaceutical or cosmetic ingredient, with one or more of these components being present within the solidified fatty substance, are further formulated into pharmaceutical or cosmetic products. The benzoyl peroxide and any other active ingredients (whether free or within the fatty substance) are stable mixed together into pharmaceutical or cosmetic skincare topical formulations, provided that the formulation is stored below the melting temperature of the fatty substance which is a temperature from about 25° C. to about 45° C., according to the precise nature of the fatty substance used, such that the benzoyl peroxide and/or other active ingredients are kept from substantially interacting with one another due to one or more of the components being substantially immobilized within the solid fatty substance and segregated from other incompatible components within the same particles of the fatty substance or different particles of the fatty substance or segregated from other incompatible components that are present outside of the fatty substance within the formulated product. When the formulation is applied and rubbed into the skin, the heat generated by the application of the formulation in combination with the natural body heat of the skin results in the solid fatty substance softening or partially or fully melting, allowing the release or diffusion of the benzoyl peroxide and any other ingredients within the fatty substance to the skin. The benzoyl peroxide and any other ingredients that are within and/or outside of the fatty substance then treat the skin or are absorbed into the skin to treat the underlying tissue. The pharmaceutical or cosmetic formulations contain benzoyl peroxide and any other ingredient in any acceptable amount, for example, from about 0.01% to about 20% benzoyl peroxide or other ingredient by weight of the pharmaceutical or cosmetic product formulation.

The pharmaceutical or cosmetic formulations optionally contain any active pharmaceutical or cosmetic ingredient discussed above (either within the fatty substance particles or within different fatty substance particles or with one or more of the components free within the final product formulated matrix whereby the fatty substance composition is present in a pharmaceutically or cosmetically acceptable carrier). The carrier can be any carrier typically used in the cosmetic or topical pharmaceutical arts. One or more adjuvants that are common in topical formulations may also be present, including water or other solvent, conventional hydrophilic or lipophilic gelling agents or thickeners; preservatives; salts; antioxidants; fragrances; emulsifiers; moisturizing agents; emollients; sequestering agents; surfactants; polymers (e.g., polyacrylic acid); basifying or acidifying agents; fillers; agents for combating free radicals; ceramides; sunscreen agents, in particular ultraviolet screening agents; insect repellents; slimming agents; colorants; bactericides; solvents; and antidandruff agents. The amounts of these various adjuvants in the formulations are those conventionally used in the fields under consideration.

Specific examples of pharmaceutical and cosmetic formulations include, without limitation, (i) topical formulations wherein the benzoyl peroxide is within the fatty substance; or (ii) topical formulations wherein the benzoyl peroxide is within the fatty substance, and wherein the formulations comprise additional pharmaceutical or cosmetic ingredients present within the formulation matrix but not within the fatty substance; or (iii) topical formulations wherein the benzoyl peroxide is within the fatty substance and wherein another active ingredient (or a plurality of active ingredients) is present in other particles of the fatty substance that are prepared separately from the particles of the fatty substance containing the benzoyl peroxide; or (iv) topical formulations wherein the benzoyl peroxide is within the fatty substance and another active ingredient is present on the surface of the fatty substance; or (v) topical formulations wherein both the benzoyl peroxide and/or the other active component or components are present in the same particles of the fatty substance but are kept separate by the solid fatty substance; or (vi) topical formulations where other active ingredient or ingredients are present in particles of fatty substance without benzoyl peroxide being present. Other examples comprise compositions similar to those listed above where the product is used for subcutaneous implantation rather than topical application.

Other examples of pharmaceutical applications comprise use of the fatty substance itself containing an active ingredient or several active ingredients but where the fatty substance has not been reduced to small particles which are then formulated with other ingredients to prepare a drug product. In this case the fatty substance containing the active ingredient or ingredients along with any other excipients or adjuvants is handled as a solid block and melted before application to the surface of the skin or under the skin, either as a topical application or as a subcutaneous application whereby the fatty substance acts as a reservoir on or under the skin to allow slow release of the pharmaceutical active ingredient from the fatty substance to the surrounding skin over a protracted period of time so as to achieve a controlled slow and sustained release of the pharmaceutical active ingredient to the surrounding tissue. Due to the nature of the fatty substance, incompatible pharmaceutical ingredients can be held within the same fatty substance particles since the components are immobilized within the fatty substance particles and therefore are substantially prevented from reacting with each other. This composition can be applied directly to the skin or can be used transdermally through the skin or can be applied subcutaneously.

The pharmaceutical and cosmetic formulations using the fatty substance in small particles containing an active ingredient or containing several active ingredients can be formulated and provided in any form appropriate for topical application, such as a lotion or serum, an aqueous gel, an oil-in-water phase (O/W) emulsion (where the fatty substance and materials within it represent the oil phase) or a water in oil phase (W/O) emulsion (where the fatty substance and materials within it represent the oil phase) with a liquid, semi-liquid, or solid consistency, such as milks, smooth creams, or pastes. These compositions are prepared according to known methods. Alternatively the fatty substance can itself comprise the main component of pharmaceutical product, handled as a melt or softened mass for preparation and dosing topically or subcutaneously.

Many of the active pharmaceutical or cosmetic ingredients discussed above are pharmaceutically acceptable salts of the active compound or can be made into pharmaceutically acceptable salts of the active compound. Generally, pharmaceutically acceptable salts of the compounds are acid-addition salts or base-addition salts that retain the biological effectiveness and properties of the compounds and are formed from suitable non-toxic organic or inorganic acids or organic or inorganic bases. Exemplary acid-addition salts include those derived from inorganic acids such as hydrochloric acid, hydrobromic acid, hydroiodic acid, sulfuric acid, sulfonic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Example base-addition salts include those derived from ammonium, potassium, sodium and, quaternary ammonium hydroxides, for example, tetramethylammonium hydroxide. Chemical modification of a pharmaceutical compound into a salt is a known technique to obtain improved physical and chemical stability, hygroscopicity, flowability and solubility of compounds. See, e.g., H. Ansel et. al., Pharmaceutical Dosage Forms and Drug Delivery Systems (6th Ed. 1995) at pp. 196 and 1456-1457.

The methods of treating skin ailments and/or diseases of the skin or underlying tissue involve administering the compositions of the present invention topically to the skin, and/or transdermally with the fatty substance deposited on the surface of the skin acting as a reservoir for slow release of the active ingredient or ingredients present within the fatty substance, and/or subcutaneously with the fatty substance acting as a reservoir for slow release of the active ingredient or ingredients. The skin ailments and/or diseases treated by the compositions of the present invention include, but are not limited to, acne, other skin bacterial infections, skin yeast infections, skin fungal infections, skin disorders caused by virus infections (such as warts, cold sores or other herpes virus infections of the skin) and skin and other cancers or other diseases of the underlying tissue, including breast cancer, melanoma and other diseases. Specifically, examples of such ailments and/or such diseases that are treated by administering the compositions of the present invention include, but are not limited to, acne, athlete's foot, rosacea, vitiligo, onychomycosis, warts, herpes infections, skin cancer such as melanoma, or disease of the underlying tissue such as breast cancer.

The following examples are provided as illustrative of the present invention and not limitative thereof. Many variations of active ingredients and fatty substances in the formulation may be made that are encompassed by the present invention.

### Example 1

A benzoyl peroxide in cocoa butter composition is prepared. Benzoyl peroxide (for example hydrous benzoyl peroxide supplied commercially by Norac Pharma)(10.0 g, 74.6% by wt), cocoa butter (3.2 g) and Tween 20 (10 mL) are added together and heated to 40-45° C. while stirring. The mixture is stirred for 1 h. Then it is poured into deionized water (90 mL) at ambient temperature. The mixture is stirred at ambient temperature overnight. After stirring, the mixture is allowed to stand for 1 h, and then the aqueous layer is decanted away. The solids are filtered and dried, resulting in a white solid (8.7 g, 72.9% benzoyl peroxide retention). The solids are analyzed by HPLC to determine the solids contain approximately 62.4% benzoyl peroxide (by weight).

### Example 2

A benzoyl peroxide in cocoa butter composition is prepared. Cocoa butter (4.0 g) and Tween 20 (12 mL) and deionized water (235 mL) are added together and heated to 40-45° C. with overhead stirring. The mixture is stirred for 1 h. Then benzoyl peroxide (25.0 g, 74.6% by wt) is added portionwise to the cocoa butter solution. The mixture is allowed to stir at 40-45° C. for 45 minutes then at ambient temperature overnight. After stirring, the mixture is allowed to stand for 1 h. Then the aqueous layer is decanted away. The solids are filtered and dried, resulting in a white solid (21.4 g, 70.0% benzoyl peroxide retention). The solids are analyzed by HPLC to determine the solids contain approximately 60.6% benzoyl peroxide (by weight).

### Example 3

A benzoyl peroxide in cocoa butter composition is prepared. A suspension of benzoyl peroxide (50.0 g, 74.6% by wt) in Tween 20 (50 mL) and water (450 mL) is stirred magnetically at 40-45° C. for 20 minutes. Cocoa butter (15.0 g) is introduced, and the suspension is stirred for an additional three hours at 40° C. The mixture is allowed to cool to ambient temperature overnight while gently stirring. The stirring is stopped to allow the solids to settle. The top layer is decanted off. The remaining solids are taken up in water (450 mL) and stirred for 1 h, and the decanting process is repeated. The solids are allowed to air dry which results in a white solid (46.3 g, 77.7% benzoyl peroxide retention). The solids are analyzed by HPLC to determine the solids contain approximately 62.6% benzoyl peroxide (by wt).

### Example 4

A benzoyl peroxide in cocoa butter composition is prepared. A suspension of benzoyl peroxide (50.0 g, 74.6% by wt) in Tween 20 (50 mL) and water (450 mL) is stirred at high shear (2000 rpm) at 40° C. for 20 minutes. Cocoa butter (30.0 g) is introduced and the suspension is stirred for one hour at 40° C. The mixture is allowed to cool to ambient temperature overnight while gently stirring with a magnetic stirrer. The stirring is stopped to allow the solids to settle. The top layer is decanted off. The remaining solids are filtered. The solids are allowed to air dry which results in a white solid (45.9 g, 83.3% benzoyl peroxide retention). The solids are analyzed by HPLC to determine the solids contain approximately 67.8% benzoyl peroxide (by wt).

### Example 5

A benzoyl peroxide in cocoa butter composition is prepared. A suspension of benzoyl peroxide (25.0 g, 74.6% by wt) in Tween 20 (25 mL) and water (225 mL) is stirred at high shear (2000 rpm) at 40° C. for 20 minutes. Cocoa butter (4.0 g) is introduced and the suspension is stirred for one hour at 40° C. The mixture is allowed to cool to ambient temperature overnight while gently stirring with magnetic stirrer. The stirring is stopped to allow the solids to settle. The top layer is decanted off. The remaining solids are filtered. The solids are allowed to air dry which results in a white solid (21.1 g, 86.0% benzoyl peroxide retention). The solids are analyzed by HPLC to determine the solids contain approximately 76.0% benzoyl peroxide (by wt).

### Example 6

A benzoyl peroxide in cocoa butter composition is prepared. A suspension of benzoyl peroxide (25.0 g, 74.6% wt.) in Tween 20 (25 mL) and water (225 mL) is stirred with an overhead stirrer (200 rpm) at 40° C. for 20 minutes. Cocoa butter (4.0 g) is introduced and the suspension is stirred for one hour at 40° C. The mixture is allowed to cool to ambient temperature overnight while gently stirring with magnetic stirrer. The stirring is stopped to allow the solids to settle. The top layer is decanted off. The remaining solids are filtered. The solids are allowed to air dry which results in a white solid (22.5 g, 91.3% benzoyl peroxide retention). The solids are analyzed by HPLC to determine the solids contain approximately 75.7% benzoyl peroxide (by wt).

### Example 7

A benzoyl peroxide and tazarotene in cocoa butter melt is prepared. Cocoa butter (1.7 g) is stirred at 40-45° C. for 20 minutes. Benzoyl peroxide (5.0 g, 74.6% by wt) is introduced and the melt is mixed for an additional 20 min, followed by tazarotene (3.3 g) and the melt is stirred for 30 min at 40° C. The mixture is allowed to cool. The solids are allowed to air dry which results in an off-white solid (10.0 g).

### Example 8

A benzoyl peroxide and tretinoin in cocoa butter melt is prepared. Cocoa butter (1.7 g) is stirred at 40-45° C. for 20 minutes. Benzoyl peroxide (5.0 g, 74.6% by wt) is introduced and the melt is mixed for an additional 20 min, followed by tretinoin (1.3 g) and the melt is stirred for 30 min at 40° C. The mixture is allowed to cool. The solids are allowed to air dry which results in an off-white solid (8.0 g).

### Example 9

A temezolomide in cocoa butter melt is prepared. Cocoa butter (5 g) is stirred at 40-45° C. for 20 minutes. Temezolomide (1.5 g) is introduced and the melt is mixed for an additional 20 min. The mixture is allowed to cool. The solids are allowed to dry which results in an off-white solid (6.5 g).

### Example 10

A dacarbazine in cocoa butter melt is prepared. Cocoa butter (5 g) is stirred at 40-45° C. for 20 minutes. Dacarbazine (1.5 g) is introduced and the melt is mixed for an additional 20 min. The mixture is allowed to cool. The solids are allowed to dry which results in an off-white solid (6.5 g).

### Example 11

A fluorouracil and methotrexate in cocoa butter melt is prepared. Cocoa butter (1.5 g) is stirred at 40-45° C. for 20 minutes. Fluorouracil (3.2 g) is introduced and the melt is mixed for an additional 20 min, followed by methotrexate (1.2 g) and the melt is stirred for 30 min at 40° C. The mixture is allowed to cool. The solids are allowed to air dry which results in an off-white solid (6.0 g).

### Example 12

Preparation of tretinoin in cocoa butter is prepared: A suspension of tretinoin (15.0 g) in Tween 20 (2.5 mL) and water (225 mL) is stirred at 40° C. for 20 minutes. Cocoa butter (8.0 g) is introduced and the suspension is stirred for one hour at 40° C. The mixture is allowed to cool to ambient temperature overnight while gently stirring with magnetic stirrer. The stirring is stopped to allow the solids to settle. The top layer is decanted off. The remaining solids are filtered. The solids are allowed to air dry which resulted in a light yellow solid (21.1 g, 92.0% tretinoin retention). The solids are analyzed by HPLC to determine the solids contained approximately 65.0% tretinoin (by wt).

### Example 13

Preparation of tazarotene in cocoa butter is prepared: A suspension of tazarotene (10.0 g) in Tween 20 (2.5 mL) and water (97.5 mL) is stirred at 40° C. for 20 minutes. Cocoa butter (2.5 g) is introduced and the suspension is stirred for one hour at 40° C. The mixture is allowed to cool to ambient temperature overnight while gently stirring with magnetic stirrer. The stirring is stopped to allow the solids to settle. The top layer is decanted off. The remaining solids are filtered. The solids are allowed to air dry which resulted in a light yellow solid (11.0 g, 88.0% tazarotene retention). The solids are analyzed by HPLC to determine the solids contained approximately 75.4% tazarotene (by wt).

### Example 14

Preparation of tolnaftate in cocoa butter is prepared: A suspension of tolnaftate (10.0 g) in Tween 20 (2.5 mL) and water (97.5 mL) is stirred at 40° C. for 20 minutes. Cocoa butter (2.5 g) is introduced and the suspension is stirred for one hour at 40° C. The mixture is allowed to cool to ambient temperature overnight while gently stirring with magnetic stirrer. The stirring is stopped to allow the solids to settle. The top layer is decanted off. The remaining solids are filtered. The solids are allowed to air dry which resulted in an off white solid.

### Example 15

Preparation of perillyl alcohol in cocoa butter is prepared: A solution of perillyl alcohol (5.0 g) in water (97.5 mL) is stirred at 40° C. for 20 minutes. Cocoa butter (2.5 g) is introduced and the suspension is stirred for one hour at 40° C. The mixture is allowed to cool to ambient temperature overnight while gently stirring with magnetic stirrer. The stirring is stopped to allow the solids to settle. The top layer is decanted off. The remaining solids are filtered. The solids are allowed to air dry which resulted in an off white solid.

### Example 16

Preparation of temezolomide in cocoa butter is prepared: A solution of temezolomide (10.0 g) in Tween 20 (2.5 mL), and water (97.5 mL) at pH <7 is stirred at 40° C. for 20 minutes. Cocoa butter (2.5 g) is introduced and the suspension is stirred for one hour at 40° C. The mixture is allowed to cool to ambient temperature overnight while gently stirring with magnetic stirrer. The stirring is stopped to allow the solids to settle. The top layer is decanted off. The remaining solids are filtered. The solids are allowed to air dry which resulted in an off white solid.

### Example 17

Preparation of adapalene in cocoa butter is prepared: A suspension of adapalene (10.0 g) in Tween 20 (2.5 mL) and water (97.5 mL) is stirred at 40° C. for 20 minutes. Cocoa butter (2.5 g) is introduced and the suspension is stirred for one hour at 40° C. The mixture is allowed to cool to ambient temperature overnight while gently stirring with magnetic stirrer. The stirring is stopped to allow the solids to settle. The top layer is decanted off. The remaining solids are filtered. The solids are allowed to air dry which resulted in an off white solid.

### Example 18

Preparation of gefitinib in cocoa butter is prepared: A suspension of gefitinib (10.0 g) in Tween 20 (2.5 mL) and water (97.5 mL) is stirred at 40° C. for 20 minutes. Cocoa butter (2.5 g) is introduced and the suspension is stirred for one hour at 40° C. The mixture is allowed to cool to ambient temperature overnight while gently stirring with magnetic stirrer. The stirring is stopped to allow the solids to settle. The top layer is decanted off. The remaining solids are filtered. The solids are allowed to air dry which resulted in an off white solid.

### Example 19

A BENZACLIN pharmaceutical composition is prepared. Polyacrylic acid (1 g), docusate sodium (0.10 g), benzoyl peroxide (0.85 g, 62.6% by wt in cocoa butter), and clindamycin (120 mg) are mixed. Water (10 mL) and 2N NaOH (1 mL) is added and the slurry is vigorously stirred.

### Example 20

A BENZAMYCIN pharmaceutical composition is prepared. Polyacrylic acid (1 g), docusate sodium (0.10 g), benzoyl peroxide (0.85 g, 62.6% by wt in cocoa butter), and erythromycin (120 mg) are mixed. Ethanol (4 mL), water (4 mL), and 2N NaOH (2 mL) are added, and the slurry is vigorously stirred.

### Example 21

A tretinoin formulation is prepared: Formulation 1—Polyacrylic acid (1 g), docusate sodium (0.10 g), benzoyl peroxide (0.85 g, 62.6% by wt in cocoa butter), and tretinoin (120 mg) are mixed. Water (10 mL) and 2N NaOH (1 mL) is added, and the slurry is vigorously mixed.

### Example 22

A tretinoin formulation is prepared: Formulation 2—stearic acid (0.5 g), isopropyl myristate (0.2 g), polyoxyl 40 stearate (0.20 g), stearyl alcohol (0.20 g), xanthan gum (0.10 g), sodium EDTA (0.10 g), benzoyl peroxide (0.85 g, 62.6% by wt in cocoa butter), propylene glycol, (0.10 g), sorbic acid (0.10 g), PPG-20 methyl glucose ether (0.10 mg), butylated hydroxytoluene (2 mg), and tretinoin (12 mg) are mixed. Ethanol (2 mL) and water (6 mL) are added, and the slurry is vigorously stirred.

### Example 23

A tazarotene formulation is prepared. Carbomer 934P (0.6 g), benzoyl peroxide (0.85 g, 62.6% by wt in cocoa butter), tazarotene (0.14 g), hexylene glycol (0.20 g), Poloxamer 407 (0.10 g), polyethylene glycol 400 (0.10 g), polysorbate 40 (0.10 g), benzyl alcohol (0.10 g), ascorbic acid (0.01 g), butylated hydroxyanisole (0.01 g), butylated hydroxytoluene (0.01 g), edentate disodium (0.02 g), and tromethamine (0.05 g) are mixed. Water (12 mL) is added, and the slurry is vigorously stirred.

### Example 24

Preparation of a pharmaceutical product containing benzoyl peroxide in cocoa butter with dapsone is prepared. A polymer thickener component is prepared by charging purified water (76.2 g) to a vessel suitable to contain 100 g of finished semi-solid product and slowly sifting Carbopol 980 (1 g) into a vortex formed by rapidly stirring the purified water. When a homogenous dispersion of Carbopol 980 and water is formed, stirring is reduced to minimize air entrapment. Benzoyl peroxide (15 g, 65% by wt. in cocoa butter) is added to the Carbopol 980 dispersion. Next, an active pharmaceutical component is prepared by charging an appropriately sized container with ethoxydiglycol (5 g) and methylparaben (0.3 g). The contents are stirred until all of the crystalline material dissolves then dapsone (0.5 g) is added. Stirring is continued until the drug dissolves.

The polymer thickener component is added to the active pharmaceutical component with mixing that immediately results in the formation of crystalline microparticles. Once the dispersion is homogenous, 10% sodium hydroxide solution (2.0 g) is added to neutralize the Carbopol 980 and form the gel.

| Component | Wt/100 g product |
|---|---|
| Dapsone | 0.5 |
| BPO (65% in cocoa butter) | 15.0 |
| Water | 76.2 |
| Carbopol 980 | 1.0 |
| Ethoxydiglycol | 5.0 |
| Methylparaben | 0.3 |
| 10% w/w aqueous sodium | 2.0 |
| hydroxide | |

### Example 25

Preparation of a pharmaceutical product containing benzoyl peroxide in cocoa butter with tazarotene is prepared. A polymer thickener component is prepared by charging purified water (20 g) followed by ascorbic acid (0.05 g) and edetate disodium (0.05 g) to a vessel suitable to contain 100 g of finished semi-solid product and slowly sifting Carbomer 934P (1.25 g) into a vortex formed by rapidly stirring the purified water. In a separate vessel is added purified water (29.25 g), benzoyl peroxide (15 g, 65% by wt. in cocoa butter) and Poloxamer 407 (0.2 g). These two solutions are mixed. In a separate vessel is added butylated hydroxytoluene (0.05 g), butylated hydroxyanisole (0.05 g), hexylene glycol (2.0 g), PEG-400 (30 g) and polysorbate 40 (0.2 g). The vessel is heated to 65° C. until all components dissolve. The solution is cooled to ambient temperature. Benzyl alcohol (1.0 g) and tazarotene (0.1 g) are added to the PEG-400 solution. The subsequent PEG-400 solution is added to the thickener component followed by neutralization with an aqueous 10% tromethamine solution.

| Component | Wt/100 g product |
|---|---|
| Tazoratene | 0.1 |
| BPO (65% in cocoa butter) | 15.0 |
| Water | 49.25 |
| Edetate disodium | 0.05 |
| Ascorbic Acid | 0.05 |
| Carbomer 934P | 1.25 |
| Poloxamer 407 | 0.2 |
| PEG-400 | 30.0 |
| Polysorbate 40 | 0.2 |
| Hexylene glycol | 2.0 |
| Butylated hydroxytoluene | 0.05 |
| Butylated hydroxyanisole | 0.05 |
| Benzyl alcohol | 1.0 |
| Tromethamine | 0.8 |

### Example 26

Preparation of a pharmaceutical product containing benzoyl peroxide in cocoa butter with tazarotene in cocoa butter is prepared. A polymer thickener component is prepared by charging purified water (20 g) followed by ascorbic acid (0.05 g) and edetate disodium (0.05 g) to a vessel suitable to contain 100 g of finished semi-solid product and slowly sifting Carbomer 934P (1.25 g) into a vortex formed by rapidly stirring the purified water. In a separate vessel is added purified water (29.25 g), benzoyl peroxide (15 g, 65% by wt. in cocoa butter) and Poloxamer 407 (0.2 g). These two solutions are mixed. In a separate vessel is added butylated hydroxytoluene (0.05 g), butylated hydroxyanisole (0.05 g), hexylene glycol (2.0 g), PEG-400 (29.5 g) and polysorbate 40 (0.2 g). The vessel is heated to 65° C. until all components dissolve. The solution is cooled to ambient temperature. Benzyl alcohol (1.0 g) and tazarotene in cocoa butter (0.15 g, 65% by wt) are added to the PEG-400 solution. The subsequent PEG-400 solution is added to the thickener component followed by neutralization with an aqueous 10% tromethamine solution.

| Component | Wt/100 g product |
|---|---|
| Tazoratene (65% in cocoa butter) | 0.15 |
| BPO (65% in cocoa butter) | 15.0 |
| Water | 49.25 |
| Edetate disodium | 0.05 |
| Ascorbic Acid | 0.05 |
| Carbomer 934P | 1.25 |
| Poloxamer 407 | 0.2 |
| PEG-400 | 30.0 |
| Polysorbate 40 | 0.2 |
| Hexylene glycol | 2.0 |
| Butylated hydroxytoluene | 0.05 |
| Butylated hydroxyanisole | 0.05 |
| Benzyl alcohol | 1.0 |
| Tromethamine | 0.8 |

### Example 27

Preparation of a pharmaceutical product containing benzoyl peroxide in cocoa butter with tretinoin is prepared. Stearic acid (17 g), xanthan gum (0.3 g), Polyoxyl 40 stearate (5 g), stearyl alcohol (3 g), isopropyl myristate (10 g), butylated hydroxytoluene (0.1 g) and purified water (49.25 g) are mixed followed by the addition of tretinoin (0.1 g). The mixture is stirred to wet and the contents disperse. Benzoyl peroxide (15 g, 65% by wt. in cocoa butter) is added and the stirring is continued to achieve a homogenous solution.

| Component | Wt/100 g product |
|---|---|
| Tretinoin | 0.1 |
| BPO (65% in cocoa butter) | 15.0 |
| Water | 49.25 |
| Xanthum gum | 0.3 |
| Polyoxyl 40 stearate | 5.0 |
| Stearic acid | 17.0 |
| Stearyl alcohol | 3.0 |
| Isopropyl myristate | 10.0 |
| Butylated hydroxytoluene | 0.1 |
| Sorbic acid | 0.2 |

### Example 28

Preparation of a pharmaceutical product containing benzoyl peroxide in cocoa butter with tretinoin in cocoa butter is prepared. Stearic acid (17 g), xanthan gum (0.3 g), Polyoxyl 40 stearate (5 g), stearyl alcohol (3 g), isopropyl myristate (10 g), butylated hydroxytoluene (0.1 g) and purified water (49.25 g) are mixed followed by the addition of tretinoin (0.15 g, 65% in cocoa butter). The mixture is stirred to wet and the contents disperse. Benzoyl peroxide (15 g, 65% by wt. in cocoa butter) is added and the stirring is continued to achieve a homogenous solution.

| Component | Wt/100 g product |
|---|---|
| Tretinoin (65% in cocoa butter) | 0.15 |
| BPO (65% in cocoa butter) | 15.0 |
| Water | 49.25 |
| Xanthum gum | 0.3 |
| Polyoxyl 40 stearate | 5.0 |
| Stearic acid | 17.0 |
| Stearyl alcohol | 3.0 |
| Isopropyl myristate | 10.0 |
| Butylated hydroxytoluene | 0.1 |
| Sorbic acid | 0.2 |

### Example 29

Preparation of a pharmaceutical product containing benzoyl peroxide in cocoa butter with adapalene is prepared. The aqueous component is prepared by charging 20.8 g of purified water followed by edetate disodium (0.05 g), sorbitan oleate (0.5 g) and glycerol (4 g) into a suitable container. In a separate container is placed propylene glycol (2 g) and sodium docusate (2 g) which is stirred until dissolution. In a third container is stirred propylene glycol (2 g), Poloxamer 124 (0.2 g), adapalene (0.1 g), benzoyl peroxide (4 g, 65% by wt. in cocoa butter) and purified water (61 g). Once the mixture of sodium docusate and propylene glycol is dissolved, the solution is mixed in with the aqueous component. The active ingredient phase is added next and the mixture is stirred until the solution is homogenous. Finally, sodium acryloyldimethyltaurate copolymer, isodecane and polysorbate 80 (Simulgel, 4.0 g) are added with mixing. Once the dispersion is homogenous, a 10% sodium hydroxide solution is added to obtain a pH 5.

| Component | Wt/100 g product |
|---|---|
| Adapalene | 0.1 |
| BPO (65% in cocoa butter) | 4.0 |
| Water | 81.1 |
| Edetate disodium | 0.1 |
| Glycerol | 4.0 |
| Propylene glycol | 4.0 |
| Sodium docusate | 2.0 |
| Poloxamer 124 | 0.2 |
| Sodium | 4.0 |
| acryloyldimethyltaurate | |
| copolymer & isodecane & | |
| polysorbate 80 | |
| Sorbitan oleate | 0.5 |
| Sodium hydroxide | pH 5 |

### Example 30

Preparation of a pharmaceutical product containing benzoyl peroxide in cocoa butter with clindamycin is prepared (BENZACLIN). Carbomer 940 (1 g), sodium docusate (2 g) and purified water (89.2 g) are mixed followed by the addition of clindamycin phosphate (0.1 g). The mixture is stirred to wet to disperse the contents. Benzoyl peroxide (7.7 g, 65% by wt. in cocoa butter) is added and the stirring is continued to achieve a homogenous solution. The pH is adjusted to 5.0-5.5 with aqueous 10% sodium hydroxide.

| Component | Wt/100 g product |
|---|---|
| Clindamycin | 0.1 |
| BPO (65% in cocoa butter) | 7.7 |
| Water | 89.2 |
| Carbomer 940 | 1.0 |
| Sodium docusate | 2.0 |
| Sodium hydroxide | pH 5.0-5.5 |

### Example 31

Preparation of a pharmaceutical product containing benzoyl peroxide in cocoa butter with tretinoin and clindamycin is prepared. Glycerin (10 g) and Tween 80 (5 g) are mixed followed by tretinoin (0.025 g). The mixture is stirred to wet and to disperse the contents. In a separate container, citric acid (0.05 g), edetate disodium (0.05 g), methylparaben (0.15 g), propylparaben (0.03 g), butylated hydroxyanisole (0.02 g) is added to purified water (68 g) in a vessel suitable to contain 100 g of finished semi-solid product. Clindamycin phosphate (1.2 g) is dissolved into the aqueous solution. Carbopol 980 (0.5 g) is added into a vortex formed by rapidly stirring the purified water. When a homogenous dispersion of Carbopol 980 is formed, stirring is reduced to minimize air entrapment. Benzoyl peroxide (15 g, 65% by wt. in cocoa butter) is added to the Carbopol 980 dispersion.

The tretinoin component is added to the Carbopol dispersion with mixing. Once the dispersion is homogenous, 10% tromethamine solution is added to obtain a pH of 5.5.

| Component | Wt/100 g product |
|---|---|
| Clindamycin Phosphate | 1.2 |
| Tretinoin | 0.025 |
| BPO (65% in cocoa butter) | 15.0 |
| Water | 68.0 |
| Edetate disodium | 0.05 |
| Butylated hydroxyanisole | 0.02 |
| Polysorbate 80 | 5.0 |
| Glycerin | 10.0 |
| Carbomer 980 | 0.5 |
| Citric acid | 0.05 |
| Methylparaben | 0.15 |
| Propylparaben | 0.03 |
| Tromethamine (10% in water) | pH 5.5 |

### Example 32

Preparation of microparticulate benzoyl peroxide suitable for use in preparing cocoa butter particles containing benzoyl peroxide of controlled particle size is prepared: A well-dispersed slurry containing approximately 10.0 wt % USP benzoyl peroxide (80 Kg, 75 wt % benzoyl peroxide (hydrous)), 0.8 wt % Poloxamer 124 (4.8 Kg), and 89.2 wt % DI water (515.2 kg) is prepared. The resulting slurry is processed through a microfluidizer (Microfluidics, Model M-110EH) at a process pressure of 20 Kpsi with the following interaction chambers installed: Chamber #1=H30Z (200 micron), Chamber #2=H10Z (100 micron). The interaction chambers and process lines between the intensifier and the product outlet are cooled with ice/water (0-4° C.). The slurry is processed through the unit in a single pass without recycling, and the micronized product collected in a single batch. The PSD of the BPM produced is characterized by a d₉₀ of 15.66 microns, a d₅₀ of 7.57 microns, and a d₁₀ of 1.76 microns.

Preparation of cocoa butter particles of controlled particle size where those cocoa butter particles contain benzoyl peroxide of controlled particle size is prepared: A well-dispersed slurry containing approximately 9.4 wt % benzoyl peroxide (d₉₀ of 15.5 microns), 0.8 wt % Poloxamer 124, 0.9% (v/v) Tween 20 and 88.9 wt % DI (deionized) water is prepared and heated to 45-50° C. While heating, the resulting slurry is processed with a Silverson L5MA mixer equipped with a fine emulsor screen. Cocoa butter is added and the suspension is processed for 2 h at 5000 rpm. This suspension is transferred to a chilled receiver which contains an equal volume of DI water. During the transfer, the internal temperature of the receiver is kept between 5-10° C. After all of the suspension is transferred, the solution is aged at 0-5° C. for at least one hour, then allowed to warm to ambient temperature overnight. A sample is taken of the slurry for PSD (particle size distribution) analysis using a MicroTrac HF particle size analyzer with DI water as the recirculation fluid. PSD d₉₀=29 microns. The suspension is filtered through a medium porosity glass frit. The resulting cake is washed with DI water then allowed to air dry.

### Example 33

The stability of the benzoyl peroxide (BP-CB) formulation of the present invention is compared with benzoyl peroxide (BPO) in non-invention drug formulations. In one study, clindamycin hydrochloride is used as the API. Clindamycin hydrochloride and BP-CB or BPO are placed in vials and stored at 30° C. The vials are analyzed by HPLC and the area from the clindamycin hydrochloride peak is monitored. After 216 h at 30° C., the HPLC analysis reveals that the area of clindamycin hydrochloride with BPO decreased 87.0% while the HPLC area of clindamycin hydrochloride with BP-E decreased 7.5%. These values are in comparison to the area from the T₀ results.

BPO Study—Into clear one dram vials is placed hydrous benzoyl peroxide (26 mg, 75.0% by wt) and clindamycin hydrochloride (20 mg). The contents are mixed thoroughly. Then the vials are stored at 30° C.

BP-CB Study—Into clear one dram vials is placed BP-CB (30 mg, 65% BPO by wt) and clindamycin hydrochloride (20 mg). The contents are mixed thoroughly then the vials are stored at 30° C.

Analysis—At certain time points, the vials are analyzed by HPLC. Prior to analysis, a mixture of methanol and acetonitrile (10 mL total, 1:1 by volume) is added to dissolve the contents of the vial. The solution is used for HPLC analysis. For T₀, a vial is removed and analyzed before the samples are heated to 30° C.

### HPLC Conditions

### Mobile Phase

### 1. Mobile Phase A: 100% DI H₂O, KH₂PO₄ (6.81 g), H₃PO₄

To prepare 1 L solution, transfer 1000 mL DI water into a suitable reservoir. Add 6.81 g of KH₂PO₄, and then adjust pH to 3.1 with H₃PO₄. Mix well. Degas solution thoroughly before use.

### 2. Mobile Phase B: 100% ACN

| Parameter | Condition | |
|---|---|---|
| Column | YMC J'sphere ODS-H80 C18, 4.6 × 150 mm, | |
| | 4-μm | |
| Flow rate | 1.5 | mL/min |
| UV Detection | 230 nm, Bw: 16, Ref: off | |
| | 210 nm, Bw: 16, Ref: off | |
| Gradient Program | See Table | |
| Column Temperature | 40° | C. |
| Injection Volume | 10 | μl |
| Run Time | 25 | min |

| Time (min) | (%) Mobile Phase A | (%) Mobile Phase B |
|---|---|---|
| 0.00 | 80.0 | 20.0 |
| 3.00 | 80.0 | 20.0 |
| 15.00 | 35.0 | 65.0 |
| 17.00 | 10.0 | 90.0 |
| 20.00 | 10.0 | 90.0 |
| 20.10 | 80.0 | 20.0 |
| 25.00 | 80.0 | 20.0 |

### Example 34

In stability testing of the benzoyl peroxide (BP-CB) of the present invention indicates that it provides greater stability with other active pharmaceutical ingredients than other benzoyl peroxide (BPO) formulations. These stability studies are set up to examine BP-CB/BPO with various APIs in aqueous gel formulations. In one study, tazarotene is used as the API. Tazarotene and BP-CB or BPO are placed into a polyacrylic acid (PAA) gel in vials and stored at 30° C. The vials are analyzed by HPLC and the area from the tazarotene peak is monitored. After 834 h at 30° C., the HPLC analysis reveals that the area of tazarotene with BPO decreases 88% while the HPLC area of tazarotene with BP-E decreases 5%. These values are compared on the area from the Tₒ results.

BPO Study—Polyacrylic acid gel is prepared by stirring polyacrylic acid (2.5 g) in deionized water (100 mL) at 50° C. for 2 h. The gel is allowed to cool to ambient temperature. The gel (500 mg) is weighed out into each of the 4-dram vials. Into the same vials is placed hydrous benzoyl peroxide (390 mg, 74.6% by wt) and tazarotene (375 mg). The contents are mixed thoroughly then the vials are stored at 30° C.

BP-CB Study—Polyacrylic acid gel is prepared by stirring polyacrylic acid (2.5 g) in deionized water (100 mL) at 50° C. for 2 h. The gel is allowed to cool to ambient temperature. The gel (500 mg) is weighed out into each of the 4-dram vials. Into the same vials is placed BP-CB (447 mg, 65% BPO by wt) and tazarotene (375 mg). The contents are mixed thoroughly then the vials are stored at 30° C.

Analysis—At certain time points, the vials are analyzed by HPLC. Prior to analysis, ethyl acetate (3 mL) is added to dissolve the contents of the vial. An aliquot (10 μL) is removed and diluted further with ethyl acetate (1.9 mL) then used for HPLC analysis. For Tₒ, a vial is removed and analyzed before the samples are heated to 30° C.

### HPLC Conditions

### Mobile Phase

### 1. Mobile Phase A: 60% DI H₂O:40% ACN+0.1% H₃PO₄

To prepare 1 L solution, transfer 600 mL DI water into a suitable reservoir. Add 400 mL ACN and then add 1 mL H₃PO₄. Mix well. Degas solution thoroughly before use.

### 2. Mobile Phase B: 5% DI H2O:95% ACN+0.1% H3PO4

To prepare 1 L solution, transfer 50 mL DI water into a suitable reservoir. Add 950 mL ACN and then add 1 mL H₃PO₄. Mix well. Degas solution thoroughly before use.

| Parameter | Condition | |
|---|---|---|
| Column | Phenomenex Luna C18 (2), 4.6 × 150 mm, 3-μm | |
| Guard Column | Phenomenex Luna C18 (2) Guard Cartridge | |
| Flow rate | 1.2 | mL/min |
| UV Detection | 230 nm, Bw: 16, Ref: off | |
| Gradient Program | See Table 2 | |
| Column Temperature | 40° | C. |
| Injection Volume | 5 | μL |
| Run Time | 45 | min |

| Time (min) | (%) Mobile Phase A | (%) Mobile Phase B |
|---|---|---|
| 0.00 | 100.0 | 0.0 |
| 3.00 | 100.0 | 0.0 |
| 30.00 | 0.0 | 100.0 |
| 35.0 | 0.0 | 100.0 |
| 36.00 | 100.0 | 0.0 |
| 45.00 | 100.0 | 0.0 |

What is claimed is:

1. A composition comprising: a first active ingredient within a fatty substance having a melting temperature of from about 25° C. to about 45° C.; wherein the fatty substance is solid at room temperature and melts or softens when applied to the skin of a subject, and wherein said first active ingredient is substantially contained within the fatty substance and prevents or mitigates adverse interaction of the first active ingredient with other ingredient(s) in the pharmaceutical product or cosmetic product in which the fatty substance containing the first active ingredient is located.

2. The composition of claim 1, wherein the first active ingredient is benzoyl peroxide.

3. The composition of claim 1, wherein the composition has particle size distribution having ad₉₀ of about 0.1 to 200 microns.

4. The composition of claim 3, wherein the composition has particle size distribution having ad₉₀ of about 10 to 50 microns.

5. The composition of claim 4, wherein the composition has particle size distribution having a d₉₀ of about 15 to 25 microns.

6. The composition of claim 1, wherein the first active ingredient and the other ingredient(s) which otherwise interact adversely with the first active ingredient are within the fatty substance and wherein the fatty substance prevents or mitigates the adverse interaction between the active ingredient(s) within it.

7. The composition of claim 2, wherein the benzoyl peroxide is deagglomerated or reduced to microparticles.

8. The composition of claim 7, wherein the composition has a benzoyl peroxide particle size distribution of d₉₀ of about 0.1 to 150 microns.

9. The composition of claim 8 wherein the composition has a benzoyl peroxide particle size distribution of d₉₀ of about 10 microns to 15 microns.

10. The composition according to any of claim 1, 2, 3, 4, 5, 6, 7, 8, or 9, wherein said other ingredient(s) and said first active ingredient is each a pharmaceutical or cosmetic ingredient(s), or a salt(s) thereof.

11. The composition of claim 1, wherein the fatty substance comprises a glycerol fatty acid ester, triglyceride, an animal, vegetable, plant or mineral fat, a synthetic fat, partially or fully dehydrogenated.

12. The composition of claim 1, wherein the fatty substance is selected from a lard, butter, palm oil, kapok, esparto, shea butter, mango butter, kokum butter, cocoa butter, decanoic acid, undecanoic acid, erucic acid, tetradeconol, trideconal, lauryl alcohol, beneicosane, mono decane, octadecane, ercosane, elemi resin, levubinic acid, palm oil, coconut oil, dimethyl sebacate, adipic acid, monoethyl ester, polyethylene glycol, diethylene glycol, monotetradecyl ether, diethylene glycol, heptaethycine glycol monododecyl ether, palmitate esters, stearate esters, polycaprolactone-block, polysetrahydro-furan-block, poly [di(ethyleneglycol) adipate], poly [trimethyl propane/diprpoylene glycol-als-adepic acid/phtholic anhydride] polyol, and mixtures thereof, olive oil, castor oil, cottonseed oil, wheat germ oil, cocoa butter, hydrogenated oils, squalane, petroleum, solid paraffin, liquid paraffin, jojoba oil, canuba wax, bees wax, lanolin, trilaurin, stearic acid, palmitic acid, capric acid, myristic acid, lauric acid, tallow fat, cochineal fat, dog grease, duck grease, palm oil, whale blubber or combination thereof.

13. The composition of claim 12, wherein the fatty substance comprises a butter derived from a plant source.

14. The composition of claim 13, wherein the fatty substance is selected from kapok, esparto, cocoa butter, shea butter, mango butter, kokum butter, or a mixture thereof.

15. The composition of claim 14, wherein the fatty substance is cocoa butter.

16. The composition of claim 10, wherein the fatty substance is selected from a glycerol fatty acid ester, triglyceryde, an animal, vegetable, plant or mineral fat, a synthetic fat, partially or fully dehydrogenated.

17. The composition of claim 16, wherein the fatty substance is selected from a lard, butter, palm oil, kapok, esparto, shea butter, mango butter, kokum butter, cocoa butter, decanoic acid, undecanoic acid, erucic acid, tetradeconol, trideconal, lauryl alcohol, beneicosane, mono decane, octadecane, ercosane, elemi resin, levubinic acid, palm oil, coconut oil, dimethyl sebacate, adipic acid, monoethyl ester, polyethylene glycol, diethylene glycol, monotetradecyl ether, diethylene glycol, heptaethycine glycol monododecyl ether, palmitate esters, stearate esters, polycaprolactone-block, polysetrahydro-furan-block, poly [di(ethyleneglycol) adipate], poly [trimethyl propane/diprpoylene glycol-als-adepic acid/phtholic anhydride] polyol, and mixtures thereof, olive oil, castor oil, cottonseed oil, wheat germ oil, cocoa butter, hydrogenated oils, squalane, petroleum, solid paraffin, liquid paraffin, jojoba oil, canuba wax, bees wax, lanolin, trilaurin, stearic acid, palmitic acid, capric acid, myristic acid, lauric acid, tallow fat, cochineal fat, dog grease, duck grease, palm oil, whale blubber or combination thereof.

18. The composition of claim 17, wherein the fatty substance comprises a butter derived from a plant source.

19. The composition of claim 18, wherein the fatty substance is selected from kapok, esparto, cocoa butter, shea butter, mango butter, kokum butter, or a mixture thereof.

20. The composition of claim 10, wherein said composition is in an aqueous-based formulation.

21. The pharmaceutical or cosmetic composition of claim 11, wherein said composition is in an aqueous-based formulation.

22. The pharmaceutical or cosmetic composition according to claim 12, wherein said composition is in an aqueous-based formulation.

23. The pharmaceutical or cosmetic composition according to claim 13, wherein said composition is in an aqueous-based formulation.

24. The pharmaceutical or cosmetic composition according to claim 14, wherein said composition is in an aqueous-based formulation.

25. The pharmaceutical or cosmetic composition according to claim 15, wherein said composition is in an aqueous-based formulation.

26. The composition according to claim 16, wherein said composition is in an aqueous-based formulation.

27. The composition according to claim 17, wherein said composition is in an aqueous-based formulation.

28. The composition according to claim 18, wherein said composition is in an aqueous-based formulation

29. The composition according to claim 19, wherein said composition is in an aqueous-based formulation.

30. The pharmaceutical or cosmetic composition of claim 10, wherein one of said active pharmaceutical or cosmetic ingredients or salts thereof is an antibiotic, a retinoid, an antifungal, a vitamin, an antiviral, a steroid, an anti-cancer drug, or a combination thereof.

31. The pharmaceutical or cosmetic formulation of any one of claim 21, 22, 23, 24, 25, 26, 27, 28, 29, or 30, wherein said active pharmaceutical or cosmetic ingredient or salt thereof is selected from an antibiotic, retinoid, an antifungal, a vitamin, an antiviral, a steroid, an anti-cancer drug, or a combination thereof.

32. The pharmaceutical or cosmetic formulation of claim 31, wherein said pharmaceutical or cosmetic ingredients or salt thereof is selected from benzoyl peroxide, bleomycin, 2,4-dinitrochlorobenzene, fluorouracil, salicyclic acid, silver nitrate, zinc sulfate, zinc oxide, cantharidin, podophyllin, imiquimod, clindamycin, clindamycin hydrochloride and clindamycin phosphate, erythromycin, tetracycline, dicloxacilin, doxycycline, minocycline, bacitracin, chlortetracycline, neomycin, mupirocin, polymyxin B, cuprimyxin, furazolidone, gentamycin, lincomycin, cephalosporins, betalactam antibiotics, lincomycin hydrochloride, tazarotene, vitamin A, retinoic acid, tretinoin, isoretinoin, adapalene, retinol, aciretin, bexarotene, retinoids; oxybutynin; vitamin D, vitamin C, vitamin B, vitamin E; sulfur; glucocorticosteroids, corticosteroids, triamcinolone, triamcinolone acetonide, betamethasone, betamethasone 17-valerate, betamethasone dipropionate, halcinonide, isoflupredone acetate, flumethasone, fluocinonide, mometasone, fluticasone, fluticasone propionate, prednisolone, beclomet(h)asone, hydrocortisone, cyproterone, drospirenone, estrogen, progestogen, tacrolimus, pimecrolimus, ursolic acid, betulinic acid, moronic acid, oleanolic acid, acyclovir, valaciclovir, famciclovir, penciclovir, docosanol, perillyl alcohol, cyclophosphamide, methotrexate, doxorubicin, paclitaxel, docetaxel, epirubicin, vemurafenib, gefitinib, anastrazole, letrozole, aromasin, tamoxifen, antiviral agents such as acyclovir, uclacyclovir, famiciclovir, penciclovir, itraconazole, fluconazole, voriconazole, ketoconazole, miconazole, miconazole nitrate, clotrimazole, sulconazole nitrate, terbinafine, econazole nitrate, tioconazole, itraconazole, posaconazole griseofulvin, nystatin, amphotericin B, neticonazole, butenafine, lanoconazole, terconazole, butoconazole, bifonazole, isoconazole, fezatione, tolnaftate, flucytosine, clioquinal, ticlatone, haloprogin, ciclopirox, natamycin, tea tree oil, selenium sulfide, acetyl salicylic acid, amorolfine, anthralin, nizoral, coal tar, resorcinol, glycolic acid, witch hazel, alpha hydroxyl acids, or combination thereof.

33. The pharmaceutical or cosmetic formulation of claim 32, wherein said active pharmaceutical or cosmetic ingredient is selected from the group consisting of benzoyl peroxide, a retinoid, or a salt thereof, tretinoin, or salt thereof, tazarotene, or salt thereof, clindamycin, or salt thereof, erythromycin, or salt thereof, or combinations thereof.

34. The pharmaceutical or cosmetic formulation of claim 33, wherein one of said active pharmaceutical or cosmetic ingredients is benzoyl peroxide.

35. The pharmaceutical or cosmetic formulation of claim 33, wherein one of said active pharmaceutical or cosmetic ingredients is tazarotene, or a salt thereof.

36. The pharmaceutical or cosmetic formulation of claim 33, wherein one of said active pharmaceutical or cosmetic ingredients is clindamycin, or a salt thereof.

37. The pharmaceutical or cosmetic formulation of claim 33, wherein one of said active pharmaceutical or cosmetic ingredients is erythromycin, or a salt thereof.

38. The pharmaceutical or cosmetic formulation of claim 33, wherein one of said active pharmaceutical or cosmetic ingredients is retinoid or a salt thereof.

39. A method for preparing a benzoyl peroxide composition, said method comprising: (a) dispersing a fatty substance with an aqueous suspension of benzoyl peroxide at a temperature of at least the melting temperature of the fatty substance in the presence of an emulsifying agent to provide a dispersion of the fatty substance; wherein the benzoyl peroxide is within the fatty substance; and wherein the fatty substance has a melting temperature of from about 25° C. to about 45° C.

40. The method of claim 39, further comprising: (b) cooling the dispersion of (a) to about room temperature to thereby solidify the fatty substance.

41. The method of claim 39, further comprising: (c) isolating the solidified fatty substance.

42. The method of claim 39, further comprising: adding a further separate active pharmaceutical or cosmetic ingredient, or salt thereof, to the fatty substance.

43. The method of claim 39, further comprising: adding a separate active pharmaceutical or cosmetic ingredient, or a salt thereof, which is not within the fatty substance.

44. The method of claim 43, wherein said active pharmaceutical or cosmetic ingredient is one or more of an antibiotic, a retinoid, an antifungal, an antiviral, a vitamin, a steroid, an anticancer, or combination thereof.

45. The method of claim 44, wherein said active pharmaceutical or cosmetic ingredient comprises one or more of: benzoyl peroxide, bleomycin, 2,4-dinitrochlorobenzene, fluorouracil, salicyclic acid, silver nitrate, zinc sulfate, zinc oxide, cantharidin, podophyllin, imiquimod, clindamycin, clindamycin hydrochloride and clindamycin phosphate, erythromycin, tetracycline, dicloxacilin, doxycycline, minocycline, bacitracin, chlortetracycline, neomycin, mupirocin, polymyxin B, cuprimyxin, furazolidone, gentamycin, lincomycin, cephalosporins, betalactam antibiotics, lincomycin hydrochloride, tazarotene, vitamin A, retinoic acid, tretinoin, isoretinoin, adapalene, retinol, aciretin, bexarotene, retinoids; oxybutynin; vitamin D, vitamin C, vitamin B, vitamin E; sulfur; glucocorticosteroids, corticosteroids, triamcinolone, triamcinolone acetonide, betamethasone, betamethasone 17-valerate, betamethasone dipropionate, halcinonide, isoflupredone acetate, flumethasone, fluocinonide, mometasone, fluticasone, fluticasone propionate, prednisolone, beclomet(h)asone, hydrocortisone, cyproterone, drospirenone, estrogen, progestogen, tacrolimus, pimecrolimus, ursolic acid, betulinic acid, moronic acid, oleanolic acid, acyclovir, valaciclovir, famciclovir, penciclovir, docosanol, perillyl alcohol, cyclophosphamide, methotrexate, doxorubicin, paclitaxel, docetaxel, epirubicin, vemurafenib, gefitinib, anastrazole, letrozole, aromasin, tamoxifen, antiviral agents such as acyclovir, uclacyclovir, famiciclovir, penciclovir, itraconazole, fluconazole, voriconazole, ketoconazole, miconazole, miconazole nitrate, clotrimazole, sulconazole nitrate, terbinafine, econazole nitrate, tioconazole, itraconazole, posaconazole griseofulvin, nystatin, amphotericin B, neticonazole, butenafine, lanoconazole, terconazole, butoconazole, bifonazole, isoconazole, fezatione, tolnaftate, flucytosine, clioquinal, ticlatone, haloprogin, ciclopirox, natamycin, tea tree oil, selenium sulfide, acetyl salicylic acid, amorolfine, anthralin, nizoral, coal tar, resorcinol, glycolic acid, witch hazel, and alpha hydroxyl acids, or combination thereof.

46. The method of claim 45, further comprising: adding a further separate active pharmaceutical or cosmetic ingredient within a further fatty substance to the solidified fatty substance; wherein the further fatty substance has a melting temperature of from about 25° C. to about 45° C.

47. The method of claim 42, wherein the salt of the active pharmaceutical ingredient dissolved therein is at a pH above the pKa of the salt.

48. The method of claim 39, wherein after step (a), the pH of the dispersion is lowered below the pKa of the salt, while mixing the dispersion, to provide droplets of the fatty substance having the active pharmaceutical ingredient therein.

49. The method of claim 39, further comprising: mixing the dispersion with a further active pharmaceutical or cosmetic ingredient, or a salt thereof.

50. A method for preparing a benzoyl peroxide composition, said method comprising: (a) dispersing a fatty substance into droplets in an aqueous suspension of benzoyl peroxide at a temperature of at least the melting temperature of the fatty substance in the presence of an emulsifying agent to provide a dispersion; wherein the benzoyl peroxide is within the fatty substance; and wherein the fatty substance has a melting temperature of from about 25° C. to about 45° C.; (b) cooling the dispersion of (a) to about room temperature to thereby solidify the fatty substance; and (c) extracting the solidified fatty substance.

51. A method for preparing a benzoyl peroxide composition, said method comprising: (a) suspending a composition comprising benzoyl peroxide in a fatty substance at a temperature of at least the melting temperature of the fatty substance to provide a suspension; wherein the fatty substance has a melting temperature of from about 25° C. to about 45° C.; and (b) forming droplets from the suspension.

52. The method of claim 50, further comprising: (c) solidifying the droplets to provide a solid composition comprising benzoyl peroxide within the fatty substance.

53. A method for preparing a benzoyl peroxide composition contained in an aqueous-based formulation, the method comprising: (a) suspending a composition comprising benzoyl peroxide in a fatty substance at a temperature of at least the melting temperature of the fatty substance to provide a suspension; wherein the fatty substance has a melting temperature of from about 25° C. to about 45° C.; (b) forming droplets from the suspension; wherein the benzoyl peroxide is within the fatty substance; and (c) solidifying the droplets to provide a solid composition.

54. A method for preparing a benzoyl peroxide composition, said method comprising: providing feedstock of benzoyl peroxide; dispersing a fatty substance at a temperature of at least the melting temperature of the fatty substance to provide a suspension, wherein the fatty substance has a melting temperature of about 25° C. to about 45° C. in water and surfactant; subjecting to high shear; then reducing the temperature below the melting temperature of the fatty substance and isolating the solid product.

55. The method of claim 54, wherein the benzoyl peroxide and active ingredient and fatty substance mixture is heated to a temperature that meets or exceeds the melting point of the fatty substance prior to subjecting it to high shear.

56. The method according to claim 55 wherein the high shear is achieved by microfluidization.

57. The method of claim 56 wherein the particle size of the fatty substance has a d₉₀ of about 0.1 to 200 microns.

58. The method of claim 57, wherein the particle size of the fatty substance has a d₉₀ of about 5 to 50 microns.

59. The method of claim 58, wherein the particle size of the fatty substance has a d₉₀ of about 15 to 30 microns.

60. A method for delivering a benzoyl peroxide composition to the skin of a subject, the method comprising: applying a composition comprising benzoyl peroxide within a fatty substance having a melting temperature of from about 25° C. to about 45° C. to the skin of a subject; wherein the fatty substance melts or softens upon application to the skin of the subject, and wherein benzoyl peroxide is substantially contained within the fatty substance and adverse interaction of benzoyl peroxide with other ingredients in the pharmaceutical or cosmetic product in which the fatty substance containing benzoyl peroxide is located is mitigated or prevented and wherein said benzoyl peroxide containing fatty substance is in an aqueous-based formulation.

61. The method of claim 60, wherein the benzoyl peroxide is about 1 to 75 wt. % of the fatty substance containing the benzoyl peroxide.

62. A method for treating diseases, said method comprising: administering the composition of claim 1 to a subject, wherein said composition is administered topically, transdermally or subcutaneously to said subject.

63. The method according to claim 62, wherein said composition is in an aqueous-based formulation.

64. The method of treating diseases according to claim 63, wherein the disease is a bacterial skin disease, a yeast skin disease, a fungal disease, a viral disease, or cancer.

65. The method of treating diseases according to claim 64, wherein the disease is acne, athlete's foot, rosacea, plantar warts, vitiligo or onychomycosis.

66. The method of treating diseases according to claim 64, wherein the cancer is skin melanoma.

67. The method of treating diseases according to claim 64, wherein the cancer is breast cancer.

68. The method of treating diseases according to claim 64, wherein said formulation provides the active ingredient(s) in a formulation that allows the active ingredient(s) to diffuse from the fatty substance in which they are contained.

69. The method of treating diseases according to claim 63, wherein said formulation is administered topically, transdermally, or subcutaneously.

70. A method for protecting air sensitive pharmaceutical or cosmetic ingredients in a formulation, said method comprising: dispersing said air sensitive pharmaceutical or cosmetic ingredients within a fatty substance that has a melting point of about 25° C. to about 45° C., wherein said fatty substance melts or softens when applied topically onto the skin of a subject or is administered transdermally or subcutaneously into the subject.

71. A method for protecting water sensitive pharmaceutical or cosmetic ingredients in a product formulation, said method comprising: dispersing said water sensitive pharmaceutical or cosmetic ingredient or ingredients within a fatty substance that has a melting temperature of between 25 C and about 45 C wherein said fatty substance may be formulated into an aqueous based pharmaceutical or cosmetic product and wherein the fatty substance melts or softens when applied topically onto the skin of a subject or is administered transdermally or subcutaneously into the subject, such that the water sensitive ingredient or ingredients is protected from the aqueous based product formulation by the fatty substance within which the water sensitive ingredient or ingredients is or are dispersed.

72. A method for minimizing exposure to active pharmaceutical ingredient or ingredients present within fatty substance through dilution of said active ingredient or ingredients within the fatty substance during handling of products formulated using fatty substance containing active ingredient or ingredients, other than at the target area for application of such products where the active ingredient can then diffuse over time from the fatty substance to the target area of skin.
